(19) United States
(12) Patent Application Publication
(10) Pub. No.: US 2024/0288419 A1
(43) Pub. Date: Aug. 29, 2024

(54) ARRAY, SOAKING SOLUTIONS AND METHOD OF SELECTING SOAKING CONDITIONS FOR SMALL MOLECULES IN BIOLOGICAL MACROMOLECULAR CRYSTALS

(71) Applicant: CRYSTALSFIRST GMBH, Marburg (DE)

(72) Inventors: Gerhard KLEBE, Marburg (DE); Stefan MERKL, Marburg (DE); Serghei GLINCA, Baunatal (DE); Kan FU, Marburg (DE)

(73) Assignee: CRYSTALSFIRST GMBH, Marburg (DE)

(21) Appl. No.: 18/025,103

(22) PCT Filed: Sep. 8, 2021
(86) PCT No.: PCT/EP2021/074734

§ 371 Date: Mar. 7, 2023

(30) Foreign Application Priority Data
Sep. 8, 2020 (EP) 20195132.4

(51) Int. Cl. G01N 33/53; C40B 30/04

(52) U.S. Cl. CPC G01N 33/5306; G01N 33/5308; C40B 30/04

(57) ABSTRACT

Subject-matter of the present invention is an array comprising soaking solutions for soaking a biological macromolecular crystal. Further, the subject of the invention is a rule-based method of selecting specific soaking solution compositions having a specific composition comprising composite solute(s) and organic solvent(s). Additionally, the subjects matter of the invention are non-aqueous soaking solutions, the soaking solutions obtained by the method of the invention and a screening method for small molecules comprising molecular probes, fragments and drug-size molecules using the soaking solutions and the use of the soaking solutions in a screening method for small molecules on a macromolecular crystal.

Subject-matter of the present invention is an array comprising soaking solutions for soaking a biological macromolecular crystal. Further, the subject of the invention is a rule-based method of selecting specific soaking solution compositions having a specific composition comprising composite solute(s) and organic solvent(s). Additionally, the subjects matter of the invention are non-aqueous soaking solutions, the soaking solutions obtained by the method of the invention and a screening method for small molecules comprising molecular probes, fragments and drug-size molecules using the soaking solutions and the use of the soaking solutions in a screening method for small molecules on a macromolecular crystal.

### BACKGROUND

Traditional methods of identifying new drug motifs in drug discovery use combinatorial chemistry and high throughput screening (HTS). This has resulted in chemical libraries of continuously increasing size and complexity, without concomitantly producing increasing numbers of lead structures. The size of the drug-like chemical universe appears to be too vast for any in vitro and in silico screening approach. A way out of this dilemma is the use of small molecules called fragments for screening. Fragments are smaller in size and weaker in both binding and activity than typical drug-size compounds. Nevertheless, fragments are sufficiently diverse and information-rich as to provide for straightforward compound development strategies.

The fragments need to be screened with techniques suitable for the detection of presumably weak interactions. Guided by such early stage structure affinity information, which is determined by i.e. the dissociation constant KD, small molecules of higher affinity can be assembled from a combination of low affinity binders or an optimization of individual low affinity binders.

Soaking is an important technique to use crystals from biological macromolecules to produce complexes from respective crystals and small molecules in crystallography-based screenings. According to the prior art a biological macromolecular crystal will be soaked in a concentrated small molecule solution. Depending on the affinity of the molecules, different concentrations of small molecule solutions are used. For low affinity molecules a higher concentrated small molecule solution is used, whereas lower concentrations of the small molecule solution can be used for high affinity molecules. During the soaking, the small molecules, which include molecular probes, fragments and drug-size molecules, will diffuse into the biological macromolecular crystal and will bind to the binding sites of the biological macromolecule. According to the prior art, the crystallization buffer of the crystallized biological macromolecule is used as soaking solution after extensive optimization. Typically, such soaking solutions comprise organic solvents such as DMSO or methanol. Normally, such organic solvents as well as small molecules and compatible solutes (osmolytes having cryoprotective properties) have a disadvantageous effect on the crystals. Notably, crystallographic measurements are either conducted at cryogenic temperatures such as at 100 K or less, or at room temperature. Crystallographic measurements using diffraction techniques (xray-, electron-, neutron diffraction) at cryogenic temperatures require the use of soaking solutions with cryoprotective features to keep an intact structure of the biological macromolecular crystal.

### PRIOR ART

Traditional methods of identifying new small molecule-drug motifs in drug discovery usually use high throughput screenings (HTS), biophysical screening cascades, phenotypic screenings, screenings on DNA-encoded libraries (DEL) etc. This has resulted in chemical libraries comprising continuously increasing numbers of small molecules, without concomitantly producing increasing numbers of lead structures. The size of the drug-like chemical universe appears to be too vast for any exhaustive in vitro and in silico screening approach. A way out of this dilemma is the use of small molecules called fragments for screening. Fragments are smaller in size and weaker in both binding and activity than typical lead-like or drug-like compounds. Nevertheless, fragments are suitable starting points for drug discovery campaigns since, once identified or validated crystallographically, they can be easily chemically modified and extended according to the geometry and biophysical setup of a protein's binding pocket. A successful extension of fragments to more drug-size compounds results in tailor-made compounds of desired affinity and molecular properties. A fragment approach proves to be suitable even in case of challenging and so called “undruggable” targets. Fragments need to be screened using techniques with suitable sensitivity for the detection of presumably weak molecular interactions. In this respect, crystallographic screening approaches are unsurpassed in terms of their sensitivity towards low affinity binders. They can discriminate binders from non-binders with very low false positive and false negative rates and, at the same time, elucidate the atomically resolved geometry of the interactions of proteins and binders.

Crystallography-based screenings for small molecules include the crystallization of biological macromolecules, especially proteins, soaking of respective crystals of biological macromolecules in solutions of small molecules, subsequent data collection using x-ray or neutron diffraction and, finally, examination of the obtained data sets in order to identify binders and elucidate binding modes. The so called “soaking” is an important technique to produce such protein-small molecule complexes. According to prior art, the experimenter usually proceeds in two steps in order to derive such soaking solution compositions. These two steps are the soaking experiment itself and the cryo-preservation of the soaked crystals.
1. The soaking experiment: For the soaking experiment the experimenter transfers according crystals from their original crystallization solution to a soaking solution. The soaking solution is usually based on the crystallization solution/buffer (or reservoir solution if the vapor diffusion method is used to produce crystals) but differs from the crystallization solution due to the presence of organic solvents that are required to dissolve and introduce small molecules targeting the biological macromolecules.
2. The cryo-preservation: For cryo-preservation according crystals are transferred from the soaking solution to a cryo-preservation solution that is usually based on the crystallization solution (or reservoir solution if the vapor diffusion method is used to produce crystals) but differs from the crystallization solution and the soaking solution due to the presence of cryoprotectants such as glycerol or polyethylene glycols and a lack of organic solvents. The cryoprotectant inhibits the appearance of water in a crystalline state (ice) in or around the crystals upon vitrification in, for example, liquid nitrogen. Vitrification is generally used in order to perform diffraction experiments at cryogenic temperatures, typically 100 K or less, in order to prevent the crystals from radiation damage in the course of the high-energetic x-ray measurements. The appearance of ice must be prevented since it can destroy the crystals and the diffraction of ice crystals superimposes the diffraction patterns of the protein crystals leading to quality loss in the obtained data sets. Measurements at room temperature are performed less often. In some cases, a special cryoprotection step is not needed due to the use of a cryoprotecting additive in the crystallization. Sometimes experts in crystallography add an amount of cryoprotectant already in the first of these two steps and leave the second step out. Like the rest of the process, this is done purely on a trial and error basis without any systematic reasoning.

If, finally, a suitable soaking and cryo-preservation solution is obtained, the actual soaking experiments are performed in the presence of a set of small molecules such as small molecule fragments. This comprises the transfer of protein crystals from the original crystallization environment (a crystallization solution) to a soaking environment, which introduces small molecules of interest dissolved in an organic solvent. Small molecules of low affinity require higher concentrations, whereas molecules of high affinity require lower concentrations of the small molecule in these solutions. In order to increase hit rates in the screenings the expert strives for highly concentrated solutions of small molecules. Since macromolecular crystals, especially proteins, in contrast to crystals of low-molecular-weight compounds, are traversed by solvent channels small molecules can diffuse easily and within seconds through crystals of adequate solvent channel size. In order to increase hit rates even for low affinity binders the expert strives for long soaking times (minutes, hours, days) since even low affinity binders can be extended towards high affinity binders by chemical modifications aiming for drug-like properties guided by medicinal chemistry. In a screening, during this soaking time the small molecules (including small molecules of different molecular weight as molecular probes, fragments, drug-size molecules) will diffuse into the macromolecule crystal, especially protein crystal, and will bind or will not bind to according binding sites. A binding site is any location of the macromolecule that builds direct or solvent mediated interactions with a respective small molecule. As drug-size small molecule-concentration a concentration of 5-20 mM or more may be used. In case of fragment-concentration concentrations of 10-50 mM or more may be used. For screening purposes in both cases concentrations as high as possible (that is: without detrimental effects on the crystals) are desired (up to 1M). However, as discussed below, one problem associated with the addition of high concentrations of small molecules dissolved in organic solvent is the impact on the stability of the crystal.

Since crystals of biological macromolecules, especially protein crystals, are very sensitive to any change in their environments, they most often exhibit a severe loss of quality or are even destroyed when they are transferred from an original crystallization solution to a soaking solution. In contrast to the original crystallization solutions, soaking solutions contain components as organic solvents and small molecules and, therefore, a soaking solution differs from the composition of the original growth/crystallization solution. Since such differences exert detrimental effects on crystals of biological macromolecules, successful crystallographic screenings require extensive optimizations of such soaking solutions in order to compensate for according detrimental effects on the crystals of biological macromolecules. According to the prior art the optimization is focused on the original crystallization solution (or reservoir solution if the crystals are produced according to the vapor diffusion-method) and requires an extensive and unsystematic trial and error search for a unique suitable soaking solution compositions that is tolerated by the individual type of crystals. Usually, the experimenter uses some kind of experimental plates in which he prepares soaking solutions that are based on the original crystallization solution (or reservoir buffer) but contain an organic solvent such as DMSO for solubilizing small molecule fragments. If the crystallization method is the vapor diffusion method, the crystallography expert usually uses a reservoir buffer in the reservoir of the experimental plates in order to maintain suitable osmotic conditions. In the approach of the prior art, the optimization is performed by applying on all possible parameters and in particular a fine-tuning of the components of the used crystallization or reservoir solution, pH-adjustments, introduction of additional additives (sometimes even cryoprotectants), changes in composition and volume ratios between reservoir buffer and soaking solution etc. and fine-tuning of the amount of organic solvent in a trial and error manner. Especially, the presence of organic solvents exerts detrimental effects on the macromolecular crystals. Therefore, this results in typically low amounts of organic solvents, typically DMSO, between 0.5% and 5%. The result is usually a single soaking solution composition that is tolerated by the crystals. After the soaking, the already mentioned cryo-preservation takes place. For the purpose of cryoprotection, again, the solution must also be optimized in order to make it tolerable for the soaked crystals. This means to introduce variations in concentrations of a variety of cryoprotectants and further adjustments as outlined above.

This approach can result in soaking conditions that reduce quality loss. However, since only one soaking condition is identified, it must be used for every soaking experiment which does not ensure best possible results in terms of hit rates, crystal diffraction and other quality parameters as mosaicity. Moreover, in the vast majority of cases, crystals of biological macromolecules are measured at cryogenic temperatures, typically 100 K or less, in order to prevent radiation damage. Measurements at room temperature are performed less often. Measurement at cryogenic temperatures requires crystal treatment using cryoprotectants and subsequent vitrification in, for example, liquid nitrogen. Again, this step of applying cryoprotective agents has a disadvantageous effect on the crystals making a re-optimization obligatory.

The pivotal point of the method and array of the present invention consists in the use of a systematic and rule-based variation of limited but critical parameters that affect crystal stability. The array and the method of the invention results in the selection and identification of not just one soaking solution but rather a range of suitable conditions that maintains and even improves crystal quality and can be utilized to compensate even for small molecule-induced effects like shifts in pH etc. A further pivotal point is that the array and the method of the invention eliminates the need to use the two distinct steps, that is, of the soaking experiment itself and the cryo-preservation. Moreover, the invention introduces a systematic rule-based process to infer suitable soaking solution compositions without the need for time-consuming, unsystematic trail & error dependent optimization steps. Using an A/B gradient algorithm to play off the general components of crs, w, os, and cs in order to find a small molecule soaking solution that is equivalent to the original crystallization solution in terms of osmotic net pressure, protein solubility, permittivity, and structural conformation where os and cs are considered to be antagonists in respect to the proteins structural conformation. Indeed, the array and the method of the invention considers the hitherto unconsidered fact that organic solvents and a class of additives known as compatible solutes compensate for detrimental effects that both components exert if used in distinct steps as in the prior art. This requires a balancing of the respective concentration of both components and very often an additional balancing to a buffer that maintains some degree of ionic strength. Furthermore, the invention allows for identifying soaking solution compositions of higher amounts of organic solvents, typically DMSO at more than 5%, preferably 10%. Advantageously, the method of the invention also often allows for very short or on the contrary extended soaking times of many hours, typically 6 to 48 hours, instead of oftentimes just seconds and minutes thus substantially improving the efficacy of the small molecule screening on the crystal. The invention thus gives more possibilities to the expert and flexibility in the setup of the small molecule screening process. In addition, in contrast with the approach of the prior art, the application of a reservoir buffer which may be oftentimes even detrimental is not required.

Furthermore, in general, it is believed that macromolecular crystals from every individual protein species require individual soaking solution compositions that are based on their original crystallization condition. One aspect of the present invention actually demonstrates that this is not true. Using the technology according to the present invention it is possible to screen rationally for soaking solution compositions that are not derived from the original crystallization solution that was used for nucleation, growth and/or storage of the respective macromolecular crystals.

In addition, the present invention allows for the determination of new soaking solutions that aim at reducing disorder in biological macromolecular crystals. Oftentimes, especially proteins contain parts of the amino acid chain that do not adopt fixed spatial configurations but are disordered. Such parts of the structure result in blurred electron densities that are difficult to subject to automated data processing. The rule-based method of the invention allows compatible solutes and organic solvents to be substantially increased while water is eliminated which, in a suitable ratio then often results in stabilization of such disordered regions. Therefore, using the rule based approach of the method of the invention, suitable soaking solutions can be determined where the crystal structure is preserved, disorder is reduced and screenings for small molecule binders can be effectively performed on the crystal.

The subject matter of the invention thus gives more possibilities to the expert and flexibility in the setup of the small molecule screening process. In addition, in contrast with the approach of the prior art, the application of a reservoir buffer which may be oftentimes even detrimental is not required.

Finally, it is assumed that soaking experiments depend on aqueous solutions. Examples that might demonstrate the opposite are product of random findings. The subject matter of the invention introduces a rule-based and systematic process to identify new water-free soaking solutions relying of the compensatory effect of compatible solutes and organic solvents that has a quality preservation effect on the crystals of biological macromolecules. The inventors shows for the first time that non aqueous soakings can be applied in screening where hydrophobic small molecules are not “soakable” (and cannot be properly dissolved) in aqueous solutions, in experiments for analyzing water required for the macromolecules structure etc.

Water exerts important roles in biological systems and interacts with biological macromolecules. Water can be divided into structure water and non-structure water. Non-structure water is disordered water in the bulk of some liquid environment and, since it is disordered, invisible in crystallographic structures. Structure water, in contrast, interacts more strongly with biological macromolecules and is known to exert important roles in folding of biological macromolecules, maintenance of this structure and the exertion of biological functions. Structure water is immobilized on the water accessible surface of a biological macromolecule. That means, that its residence time at respective locations is significantly longer than that of other water species. This is due to the establishment of hydrogen bonds or due to electrostatic interactions to or with the biological macromolecule. Because of this longer residence times structure water is ordered within a crystal and therefore visible in crystallographic models. Nevertheless, there are differences among structure waters in terms of the strength of the bonds to the biological macromolecule. Conditioning of biological macromolecules in water-free (also equivalently referred herein as non-aqueous) environments can elucidate which structure waters do bind strongly to the biological macromolecule and which ones do bind weakly. This is because conditioning in water-free environments removes disordered water from the biological macromolecule's crystals and even some structure water that belongs to a weakly binding fraction. Therefore, conditioning of macromolecular crystals in water-free environments elucidates the fraction of water that binds strongly to the biological macromolecule and therefore, most likely, is obligatory for structure maintenance. The remaining waters are removed from the crystal's solvent channels and the positions of formerly weakly bound structure waters are occupied by components of the water-free environment as for example DMSO. Comparing the waters visible in macromolecular structures of aqueous environments and waters and for example DMSO molecules, respectively, in macromolecular structures from water-free environments results in the identification of the fraction of water that is assumed necessary for structure maintenance and water that is not. Since water molecules have an important role in ligand binding the result of this analysis can provide important insights into water that can be utilized in order to improve binding of compounds that are meant to exert a pharmacological effect on the according biological macromolecule.

### DESCRIPTION

Subject-matter of the present invention is an array for soaking of biological macromolecular crystals, a method for selecting a suitable non-aqueous soaking solution and the composition of the soaking solution and a method of small molecule screening using the soaking solutions selected with the method of the invention.

The array of the invention comprising a first dimension of at least two individual non-aqueous soaking solutions (1n to xn) and a second dimension of at least two individual non-aqueous soaking solutions (1m to ym) wherein each of said soaking solutions is located in a separated compartment of said array, and wherein each of said soaking solution comprises an organic solvent (os) and a compatible solute (cs).

Subject matter of the present invention is an array according to the present invention, wherein the total volume of each of the individual non-aqueous soaking solutions is usually in a range of μm to ml, typically between 2.5 μl and 10 μl.

Unless the volume is expressed specifically in standard measures of volume e.g. μm, ml, “V” such as in Vos and Vcs refers to the proportion/percentage of the relevant component of the solution to the total volume (VT) of the solution i.e. 100%. For instance Vos refers to the percentage/proportion of organic solvent (os) in the whole volume of solution e.g. Vos=10 means os is 10% of the total volume of the solution.

Ncs refers to the number of moles of compatible solute (cs) and Nos refers to the number of moles of organic solvent (os).

The proportion of liquid compatible solute (cs) to organic solvent (os) may be defined either through the ratio of volume compatible solute Vcs to volume organic solvent Vos, that is Vcs:Vos, or, alternatively, through the concentration Ccs in mol per liter or number of moles, respectively, of compatible solute Ncs per volume organic solvent Vos, that is Ccs=Ncs:Vcs. Both definitions are considered equivalent within the invention. The proportion of a solid compatible solute (cs) to organic solvent (os) may be defined through the concentration Ccs or number of moles compatible solute Ncs per volume organic solvent Vos, respectively.

Alternatively, the proportion of organic solvent (os) to compatible solute (cs) may be defined through the molar ratio of organic solvent (os) to compatible solute (cs) Mos/cs=Ncs:Nos.

Depending on the required precision in a given array the preparation of the solution e.g. by pipetting technique may differ. If a high precision is required, low volumes in a pico- to microliter range can be pipetted by automated pipetting (i.e. a pipetting robot). However, for logical reasons volumes in a micro-milliliter range are preferably used in the array as they can be pipetted by hand (i.e. by technical assistance). Nevertheless, the volume that can be used in the array is only limited by the plate (custom made in a i.e. 3D printer or commercially available) in which the soaking is performed.

In a particular embodiment of the invention the soaking time is in a range of seconds to 24 months, preferably to 52 weeks, more preferably 72 hours. Preferably, the soaking time is from 6 hours to 24 hours.

Subject matter of the present invention is an array according to the present invention wherein the compatible solute is selected from the group comprising polyols, amino acids, methylamines and other compatible solutes.

In a particular embodiment of the invention the array comprises a compatible solute wherein the polyols are selected from a group comprising poly(oxyethylene), monosaccharide, disaccharide, trisaccharide, sugar alcohols, cyclitols and derivatives of former molecules.

Mixtures of any of the compatible solutes can also be used.

In a further particular embodiment of the present invention the group of poly(oxyethylene) comprises monodisperse or polydisperse poly(oxyethylene) of molecular weights ranging between 20-2000000 g/mol and includes but is not limited to for example polyethylene glycol 400, propane-1,2,3-triol (glycerol), ethane-1,2-diol (ethylene glycol), 2-Methylpentane-2,4-diol (2-Methylpentane-2,4-diol),(3R,4S,5S,6R)-2-(2,3-dihydroxypropoxy)-6-(hydroxymethyl)oxane-3,4,5-triol (1-Glucosylglycerol).

In another further particular embodiment of the present invention the group of monosaccharides comprises preferably but is not limited to i.e. (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside).

In an additional particular embodiment of the present invention the group of disaccharides comprises preferably but is not limited to i.e. (β-D-Fructofuranosyl α-D-glucopyranoside (sucrose), (2R,3S,4S,5R,6R)-2-(Hydroxymethyl)-6-[(2R,3R,4S,5S,6R)-3,4,5-trihydroxy-6-(hydroxymethyl)oxan-2-yl]oxyoxane-3,4,5-triol (trehalose).

In another particular embodiment of the present invention the group of trisaccharides comprises preferably but is not limited to i.e. (2R,3R,4S,5S,6R)-2-[(2S,3S,4S,5R)-3,4-dihydroxy-2,5-bis(hydroxymethyl)oxolan-2-yl]oxy-6-[(2S,3R,4S,5R,6R)-3,4,5-trihydroxy-6-(hydroxymethyl)oxan-2-yl]oxymethyl]oxane-3,4,5-triol (raffinose).

In another particular embodiment of the present invention the group of sugar alcohols comprises preferably but is not limited to i.e. (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol) and (2R,3S,4R,5S)-hexane-1,2,3,4,5,6-hexol (galactitol)).

In another embodiment of the present invention the group of cyclitols comprises preferably but is not limited to i.e. (1S,2S,4S,5R)-6-methoxycyclohexane-1,2,3,4,5-pentol (pinitol) and (2S,3R,4S,5R,6R)-2-[(1,3-dihydroxypropan-2-yl)oxy]-6-(hydroxymethyl)oxane-3,4,5-triol (fluoridoside).

In an additional particular embodiment of the present invention the group of derivatives of former molecules comprises preferably but is not limited to i.e. mannose derivatives as fiorin ((2R)—O2-(β-D-mannopyranosyl)glyceric acid) and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose. (In all possible stereoisomers).

In another particular embodiment of the invention the array comprises a compatible solute wherein the polyols are selected from a preferred group comprising poly(oxyethylene) of molecular weights ranging between 200-20000 g/mol and includes but is not limited to poly(oxyethylene) (200 g/mol, 300 g/mol, 400 g/mol, 550 g/mol, 600 g/mol, 1000 g/mol, 1500 g/mol, 2000 g/mol, 3350 g/mol, 4000 g/mol, 5000 g/mol, 6000 g/mol, 8000 g/mol, 10000 g/mol, 20000 g/mol), propane-1,2,3-triol (glycerol), ethane-1,2-diol (ethylene glycol), 2-Methylpentane-2,4-diol (2-Methylpentane-2,4-diol), (3S,4R,5R)-1,3,4,5,6-Pentahydroxyhexan-2-one (fructose), (2R,3S,4R,5R)-2,3,4,5,6-Pentahydroxyhexanal (glucose), (2S,3R,4R,5R)-Hexane-1,2,3,4,5,6-hexol (sorbitol), (β-D-Fructofuranosyl α-D-glucopyranoside (sucrose), (2R,3S,4S,5R,6R)-2-(Hydroxymethyl)-6-[(2R,3R,4S,5S,6R)-3,4,5-trihydroxy-6-(hydroxymethyl)oxan-2-yl]oxyoxane-3,4,5-triol (trehalose)), inositol derivatives (preferably di-myoinositol-1,1′-phosphate).

In a further particular embodiment of the invention the array comprises a compatible solute wherein the polyols are selected from a more preferred group comprising poly(oxyethylene) of molecular weights ranging between 400-20000 g/mol and includes but is not limited to poly(oxyethylene) (400 g/mol, 550 g/mol, 3350 g/mol, 6000 g/mol, 8000 g/mol, 10000 g/mol, 20000 g/mol), propane-1,2,3-triol (glycerol), ethane-1,2-diol (ethylene glycol), 2-Methylpentane-2,4-diol (2-Methylpentane-2,4-diol), β-D-Fructofuranosyl α-D-glucopyranoside (sucrose), (2R,3S,4R,5R)-2,3,4,5,6-Pentahydroxyhexanal (glucose), (2S,3R,4R,5R)-Hexane-1,2,3,4,5,6-hexol (sorbitol), (2R,3S,4S,5R,6R)-2-(Hydroxymethyl)-6-[(2R,3R,4S,5S,6R)-3,4,5-trihydroxy-6-(hydroxymethyl)oxan-2-yl]oxyoxane-3,4,5-triol (trehalose).

In an additional particular embodiment of the invention the array comprises a compatible solute wherein the polyols are selected from a most preferred group comprising poly(oxyethylene) of molecular weights ranging between 400-20000 g/mol and includes but is not limited to poly(oxyethylene) (400 g/mol), propane-1,2,3-triol (glycerol), ethane-1,2-diol (ethylene glycol), 2-Methylpentane-2,4-diol (2-Methylpentane-2,4-diol), (2R,3S,4S,5R,6R)-2-(Hydroxymethyl)-6-[(2R,3R,4S,5S,6R)-3,4,5-trihydroxy-6-(hydroxymethyl)oxan-2-yl] oxyoxane-3,4,5-triol (trehalose), (2S,3R,4R,5R)-Hexane-1,2,3,4,5,6-hexol (sorbitol), ß-D-Fructofuranosyl α-D-glucopyranoside (sucrose). Mixtures of compatible solute such as poly(oxyethylene) can also be used.

According to the present invention the group of amino acids (amino-group in alpha and beta position from the carboxylate group) comprises preferably 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), β-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[(1R)-1-carboxyethyl]amino]-5-(diaminomethylideneamino)pentanoic acid (Octopin). It shall be noted that the above mentioned amino acids selected from the preferred group can occur in all possible stereoisomers. Mixtures of any of the above compatible solutes can also be used.

According to the present invention the group of methylamines comprises preferably N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine).

According to the present invention the group of other compatible solutes comprises: 1-methyl-2-pyridinecarboxylic acid (homarine), methylsulfonium solutes as 3-dimethylsulfoniopropanoate (Dimethylsulfoniopropionate), and methylsulfinylmethane as dimethyl sulfoxide, urea (and derivatives) and other compounds with traits of compatible solutes according to the definition.

Subject matter of the present invention is an array according to the present invention wherein the compatible solute is selected from the most preferred group comprising: PEG400, MPD, EG and glycerol.

According to the present invention, mixtures of single compatible solutes selected from different groups and the same group of the compatible solutes comprising polyols, amino acids, methylamines and other compatible solutes can be used in the array. Thus, the term compatible solutes according to the present invention may refer to a mixture of compatible solutes. In a preferred embodiment, cs is a mixture of PEG, preferably PEG400 and PEG3350.

A preferred embodiment according to the present invention is a method of selecting the composition of a soaking solution, wherein the compatible solute is selected from the group comprising polyols, amino acids, methylamines or mixtures thereof, wherein the polyols are selected from a group comprising poly(oxyethylene), monosaccharide, disaccharide, trisaccharide, sugar alcohols, cyclitols and derivatives of former molecules, and wherein the amino acids are selected from a group comprising 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), β-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[(1R)-1-carboxyethyl]amino]-5-(diaminomethylideneamino)pentanoic acid (Octopin) and wherein the methylamines are selected from a group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine).

Another preferred embodiment according to the present invention is a method of selecting the composition of a soaking solution of claim 1 or 2, wherein the compatible solute is selected from the group comprising polyols, amino acids, methylamines or mixtures thereof, wherein the polyols are selected from a group comprising poly(oxyethylene), polyhydric alcohols, monosaccharide, disaccharide, trisaccharide, sugar alcohols, cyclitols and derivatives of former molecules wherein the group of poly(oxyethylene) is selected from a group comprising monodisperse or polydisperse poly(oxyethylene) of molecular weights ranging between 20-2000000 g/mol and includes but is not limited to for example polyethylene glycol 200, polyethylene glycol 400, polyethylene glycol 600, and wherein the group of polyhydric alcohols is selected from a group comprising propane-1,2,3-triol (glycerol), ethane-1,2-diol (ethylene glycol), 2-Methylpentane-2,4-diol (2-Methylpentane-2,4-diol), (3R,4S,5S,6R)-2-(2,3-dihydroxypropoxy)-6-(hydroxymethyl)oxane-3,4,5-triol (1-Glucosylglycerol) and wherein the group of monosaccharide is selected from the group comprising (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside) and wherein the group of disaccharides is selected from the group comprising β-D-Fructofuranosyl α-D-glucopyranoside (sucrose), (2R,3S,4S,5R,6R)-2-(Hydroxymethyl)-6-[(2R,3R,4S,5S,6R)-3,4,5-trihydroxy-6-(hydroxymethyl)oxan-2-yl]oxyoxane-3,4,5-triol (trehalose) and wherein the group of trisaccharides is selected from the group comprising (2R,3R,4S,5S,6R)-2-[(2S,3S,4S,5R)-3,4-dihydroxy-2,5-bis(hydroxymethyl)oxolan-2-yl]oxy-6-[(2S,3R,4S,5R,6R)-3,4,5-trihydroxy-6-(hydroxymethyl)oxan-2-yl]oxymethyl]oxane-3,4,5-triol (raffinose) and wherein the group of sugar alcohols comprises (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol) and (2R,3S,4R,5S)-hexane-1,2,3,4,5,6-hexol (galactitol)) and wherein the group of cyclitols comprises (1S,2S,4S,5R)-6-methoxycyclohexane-1,2,3,4,5-pentol (pinitol) and (2S,3R,4S,5R,6R)-2-[(1,3-dihydroxypropan-2-yl)oxy]-6-(hydroxymethyl)oxane-3,4,5-triol (fluoridoside) and wherein the group of derivatives of former molecules comprises mannose derivatives as fiorin ((2R)—O2-(β-D-mannopyranosyl)glyceric acid) and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose.

In one embodiment of the above the described array, the array comprises a first dimension of a series of at least two individual non-aqueous soaking solutions for the soaking of a biological macromolecule wherein the compatible solute of the second dimension of a series of at least two individual soaking solutions differs from the one of the first dimension. In one embodiment, the described array, the array comprises of 1x to xn series in said first dimension of individual soaking solutions for the biological macromolecule wherein the compatible solute of each of the series of individual non-aqueous soaking solution in the first dimension differs from the one of the series of individual soaking solutions of the second dimension.

In one embodiment, the described array, the array comprises of 1x to xn series in said first dimension of individual soaking solutions for the biological macromolecule wherein organic solvent of each of the series of individual non-aqueous soaking solution in the first dimension differs from the one of the series of individual soaking solutions of the second dimension.

In one embodiment, the described array, the array comprises of 1x to xn series in said first dimension of individual soaking solutions for the biological macromolecule wherein the compatible solute of each of the series of individual non-aqueous soaking solution in the first dimension differs from the one of the series of individual soaking solutions of the second dimension and the organic solvent of each of the series of individual non-aqueous soaking solution in the first dimension differs from the one of the series of individual soaking solutions of the second dimension.

Subject matter of the present invention is an array according to the present invention, wherein the organic solvent comprises liquid carbohydrates, protic or aprotic, of low reactivity, that can serve to solve small molecules. The examples of the organic solvents include, but are not limited to: straight or branched chain monohydric aliphatic alcohols, such as methanol, ethanol, propan-1-ol, propan-2-ol, 2-methylpropan-1-ol (isobutanol), butan-1-ol (n-butanol), 2-methylpropan-2-ol (tert-butanol); di- or polyhydric aliphatic alcohols, such as 2-methyl-2,4-pentanediol hexane-2,5-diol (2,5-Hexanediol), propane-1,3-diol (1,3-propanediol), butane-2,3-diol, propane-1,2-diol (1,2-propanediol), ethane-1,2-diol (ethylen glycol); monoalkyl ethers of an aliphatic dihydric alcohols, such as monomethyl ether of ethylene glycol; dialkyl ketones, such as propan-2-one (acetone); cyclic non-aromatic ethers, such as oxolane (tetrahydrofuran), crown ethers, 1,4-dioxane (dioxane); cyclic enol-ethers, such as furan; benzyl alcohols; phenyl ethyl alcohols; (alkylated) sulfoxides, such as methylsulfinylmethane (DMSO) and its derivatives; esters derived from acetic acids, such as ethyl acetate and its derivatives; alkylated formamides, such as N,N-dimethylformamide; organic nitriles, such as acetonitrile; lactones such as oxolan-2-one (gamma-butyrolactone), 4-methyloxetan-2-one (beta-butyrolactone); polyethers, such aspolyethylene glycol (for example 400); polyetheramines, such as Jeffamine (for example 400); glycolethers, such as 2-(2-ethenoxyethoxy)ethanol (diethylene glycol); and others suitable to dissolve small molecules for protein crystal-based screenings.

In another embodiment instead of a cs a salt is combined with os according to the described procedure wherein the salt is selected from the group comprising disodium;2,3-dihydroxybutanedioate (sodium tartrate), (2R,3R)-2,3-dihydroxybutanedioate (dipotassium tartrate), azanium;phosphates (ammonium phosphates), diazanium; sulfate (ammonium sulfate), triazanium;2-hydroxypropane-1,2,3-tricarboxylate (ammonium citrate), sodium; acetate (sodium ethanoate), azanium; acetate (ammonium acetate), dilithium;sulfate, lithium; chloride, lithium; acetate, lithium; formate, lithium; nitrate, sodium;nitrate, sodium;chloride, potassium; chloride, sodium; formate, monophosphate from sodium and potassium, di- and polyphosphates from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate)), magnesium;diformate, magnesium;dichloride, magnesium;sulfate, calcium; dichloride; dihydrate, disodium; butanedioate (sodium succinate), cadmium(II) sulfate (cadmium sulfate), disodium;propanedioate (sodium malonate), magnesium;diacetate (magnesium acetate), zinc;diacetate (zinc acetate), calcium;diacetate (calcium acetate) and wherein the salt is preferably selected from the group lithium;acetate, lithium;chloride, lithium;formate, lithium; nitrate, dilithium;sulfate, sodium;chloride, potassium; chloride, magnesium;dichloride, diazanium; sulfate (ammonium sulfate), sodium;acetate (sodium ethanoate), sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium; diformate, sodium; nitrate.

In one embodiment of the invention, method of selecting the composition of a soaking solution, wherein said soaking solution comprises an organic solvent (os) and a compatible solute (cs),
wherein said compatible solute (cs) is a polyol or a mixture thereof, and said organic solvent (os) is a liquid carbohydrate protic or aprotic, of low reactivity, or a mixture thereof, that can serve to solve small molecules,
more preferably, said compatible solute (cs) is a poly(oxyethylene) and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute cs is selected from the group comprising the poly(oxyethylene)s polyethylene glycole 200, polyethylene glycol 400, polyethylene glycole or a mixture thereof and said organic solvent (os) is selected from the group comprising dimethyl sulfoxide, 1,4-dioxane, dimethylformamide, methanol, ethanol, or a mixture thereof.

In one embodiment of the invention, method of selecting the composition of a soaking solution, wherein said soaking solution comprises an organic solvent (os) and a compatible solute (cs),
wherein said compatible solute (cs) is a polyol or a mixture thereof, and said organic solvent is a is a liquid carbohydrate protic or aprotic, of low reactivity, or a mixture thereof, that can serve to solve small molecules,
more preferably, said compatible solute (cs) is a polyhydric alcohol and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute (cs) is selected from the group comprising the polyhydric alcohols propane-1,2,3-triol (glycerol), ethane-1,2-diol (ethylene glycol), 2-Methylpentane-2,4-diol (MPD), (3R,4S,5S,6R)-2-(2,3-dihydroxypropoxy)-6-(hydroxymethyl)oxane-3,4,5-triol (1-Glucosylglycerol) or a mixtures thereof, and said organic solvent (os) is selected from the group comprising dimethyl sulfoxide, 1,4-dioxane, dimethylformamide, methanol, ethanol, or a mixture thereof.

In one embodiment of the invention, method of selecting the composition of a soaking solution, wherein said soaking solution comprises an organic solvent (os) and a compatible solute (cs),
wherein said compatible solute (cs) is a polyol a or a mixture thereof, and said organic solvent (os) is a liquid carbohydrate protic or aprotic, of low reactivity, or a mixture thereof, that can serve to solve small molecules,
more preferably said compatible solute (cs) is a mono-, di-, tri, oligo- or polysaccharide or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute (cs) is selected from the group comprising the saccharides (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) or β-D-Fructofuranosyl α-D-glucopyranoside (sucrose) or a mixtures thereof, and said organic solvent (os) is selected from the group comprising dimethyl sulfoxide, 1,4-dioxane, dimethylformamide, methanol, ethanol, or a mixture thereof.

In one embodiment of the invention, method of selecting the composition of a soaking solution, wherein said soaking solution comprises an organic solvent (os) and a compatible solute (cs),
wherein said compatible solute (cs) is a L- or D-amino acid, or a mixture thereof, and the organic solvent (os) is liquid carbohydrate protic or aprotic, of low reactivity, or a mixture thereof, that can serve to solve small molecules,
more preferably said compatible solute (cs) is a L-amino acid, or a mixture thereof, and said organic solvent is a (alkylated) sulfoxide, cyclic non-aromatic ether, straight or branched chain monohydric aliphatic alcohol, alkylated formamide, or a mixture thereof,
most preferably, said compatible solute (cs) is selected from the group comprising the L-amino acids (2S)-pyrrolidine-2-carboxylic acid (proline), 2-aminoacetic acid (glycine), (6S)-2-methyl-1,4,5,6-tetrahydropyrimidine-6-carboxylic acid (ectoine) or (5S,6S)-5-hydroxy-2-methyl-1,4,5,6-tetrahydropyrimidine-6-carboxylic acid (hydroxyectoine), or a mixture thereof, and said organic solvent (os) is selected from the group comprising dimethyl sulfoxide, 1,4-dioxane, dimethylformamide, methanol, ethanol, or a mixture thereof.

In one embodiment of the invention, method of selecting the composition of a soaking solution, wherein said soaking solution comprises an organic solvent (os) and a compatible solute (cs), wherein said compatible solute (cs) is a methylamine or a mixture thereof and said organic solvent (os) is liquid carbohydrate protic or aprotic, of low reactivity, or a mixture thereof that can serve to solve small molecules,
more preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is a (alkylated) sulfoxide, cyclic non-aromatic ether, straight or branched chain monohydric aliphatic alcohol, alkylated formamide, or a mixture thereof,
most preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is selected from the group comprising dimethyl sulfoxide, 1,4-dioxane, dimethylformamide, methanol, ethanol, or a mixture thereof.

In one embodiment of the invention, method of selecting the composition of a soaking solution, wherein said soaking solution comprises an organic solvent (os) and a compatible solute (cs), and wherein said compatible solute (cs) is polyethylene glycole and said organic solvent (os) is dimethyl sulfoxide.

In one embodiment of the invention, method of selecting the composition of a soaking solution, wherein said soaking solution comprises an organic solvent (os) and a compatible solute (cs), wherein said compatible solute (cs) is ethane-1,2-diol (ethylene glycol) and said organic solvent (os) is dimethyl sulfoxide.

In one embodiment of the invention, method of selecting the composition of a soaking solution, wherein said soaking solution comprises an organic solvent (os) and a compatible solute (cs), wherein said compatible solute (cs) is propane-1,2,3-triol (glycerol) and said organic solvent (os) is dimethyl sulfoxide.

In one embodiment of the invention, method of selecting the composition of a soaking solution, wherein said soaking solution comprises an organic solvent (os) and a compatible solute (cs), wherein said compatible solute (cs) is 2-methylpentane-2,4-diol (MPD) and said organic solvent (os) is dimethyl sulfoxide.

According to the present invention, mixtures of single components of the different constituents of the soaking solution selected from different groups and the same group of the respective single components of the soaking solution comprising the organic solvent and a compatible solute can be used in the array.

In a particular embodiment of the present invention the array according to the present invention comprises each dimension of individual soaking solutions is physically arranged in dimension dl of said array. The dimension dl of individual soaking solutions may or may not be arranged physically in a row of an array. It could be also arranged differently as long as the position of each individual soaking solution may be determined bijective, i.e. in a one-to-one correspondence.

Subject matter of the present invention is the use of an array according to the present invention for testing the integrity of a biological macromolecular crystal or the use of an array according to the present invention for testing a biological macromolecular crystal regarding tolerance towards a) changes in the original growth environment by the introduction of organic solvents, compatible solutes (especially typical cryoprotectants) b) stress caused by i.e. changes in the pH value and/or changes in osmotic pressure in the growth environment of the crystal by introduction of i.e. organic solvents, compatible solutes, and/or small molecules or by other individual properties of the used substances (organic solvents, small molecules, etc. added to conditions evaluated in a), c) or the use of an array according to the present invention for a small molecule screening process of a biological macromolecular crystal or d) the use of an array according to the present invention for identifying specific soaking solution compositions of said array that is suitable for a small molecule screening process of said biological macromolecular crystal or e) the use of an array according to the present invention for identifying specific soaking solution compositions of said array that is suitable for reducing disorder in disordered regions of the biological macromolecule.

In another aspect, the subject matter of the invention is the use of a non-aqueous soaking solution for soaking the crystal form of a biological macromolecule, for testing the integrity of a biological macromolecular crystal or for a screening process of a biological macromolecular crystal or for identifying a specific soaking solution composition that is suitable for a screening process of said biological macromolecular crystal, wherein the non-aqueous soaking solution comprises at least an organic solvent (os) and at least a compatible solute (cs).

In another aspect of the invention, the method of the invention is a method of selecting the composition of a non-aqueous soaking solution suitable for soaking the crystal form of a biological macromolecule wherein the solution comprises an organic solvent (os) and a compatible solute (cs) comprising the steps of
1) preparing the soaking solution by mixing os and cs
2) transferring at least one crystal of the biological macromolecular crystal into the solution prepared in step 1) or transferring each solution prepared in step 1) into a compartment comprising at least one crystal of the biological macromolecular crystal
3) controlling the crystal
4) selecting the soaking solution which is suitable for soaking the crystal.

A most preferred embodiment of the present invention is a method of selecting the composition
of a non-aqueous soaking solution suitable for soaking the crystal form of a biological macromolecule wherein the solution comprises an organic solvent (os) and a compatible solute (cs) comprising the steps of:
   1) preparing the soaking solution by mixing os and cs
   2) transferring at least one crystal of the biological macromolecular crystal into the solution prepared in step 1) or transferring each solution prepared in step 1) into a compartment comprising at least one crystal of the biological macromolecular crystal
   3) controlling the crystal
   4) selecting the soaking solution which is suitable for soaking the crystal; and
wherein the compatible solute is selected from the group comprising polyols, amino acids, methylamines, and mixtures thereof, and
wherein the organic solvent is selected from the group comprising liquid carbohydrates, protic or aprotic, of low reactivity, that can serve to solve small molecules and, mixtures thereof.

In a further embodiment, the method of the invention is a method of selecting the composition of a non-aqueous soaking solution for soaking the crystal form of a biological macromolecule wherein the soaking solution comprises an organic solvent (os) and a compatible solute (cs); comprising the steps of
1) preparing a first (also called benchmark) series of 1n to xn individual soaking solutions by mixing os and cs, wherein:
a) x is at least 2
b) each soaking solutions of the first series comprises the same organic solvent (os) and the same compatible solute (cs)
c) Vos and Vcs are changed in each solution of the series, and
   in the solution 1n, Vos is the minimum Vos (Vosmin) and the Vcs is the maximum Vcs (Vcsmax), and
   in the solution xn, Vcs is the minimum Vcs (Vcsmin) and the Vos is the maximum Vos (Vosmax), and
   when x>2, in any additional solutions between 1n and xn, the Vos is varied between Vosmin and Vosmax and, inversely, Vcs is varied between Vcsmin and Vcsmax;
   or, alternatively, Vos and Ncs are changed in each solution of the series, and
      in the solution 1n, Vₒₛ is the minimum Vₒₛ (Vosmin) and the N_{cs} is the maximum N_{cs} (N_{cs}max), and
      in the solution xn, Nos is the minimum Nos (N_{cs}min) and the Vₒₛ is the maximum Vₒₛ (Vₒₛmax), and
      when x>2, in any additional solutions between 1n and xn, the Vₒₛ is varied between Vosmin and Vₒₛmax and, inversely, N_{cs} is varied between N_{cs}min and N_{cs}max,
2) transferring at least one crystal of the biological macromolecular crystal into y compartments, each compartment comprising one of the solutions prepared in step 1 or transferring each solution prepared in step 1 into y compartments, each compartment comprising at least one crystal of the biological macromolecular crystal from its crystallization solution.
3) controlling the crystal in each compartment
4) selecting the soaking solution(s) which is/are suitable for soaking the crystal.

In a preferred embodiment, the soaking solutions of the first or benchmark series are distributed in each compartment to form a first dimension. Typically, the first dimension is a line or a column of wells in a screening array.

In a most preferred embodiment, the method of selecting the composition of a non-aqueous soaking solution further comprises the step of
1) preparing m series of 1n to yn individual soaking solutions by mixing os and cs wherein m is at least 1 and wherein in each of the m series
a) y is at least 2
b) os is the same as in the first (benchmark) series, and
c) the cs is changed and is different from the cs of the first(benchmark) series, and wherein in each individual solutions of each of the m series
d) Vos and Vcs are changed in each solution, and
   in the solution 1n, Vos is the minimum Vos (Vosmin) and the Vcs is the maximum Vcs (Vcsmax), and
   in the solution yn, Vcs is the minimum Vcs (Vcsmin) and the Vos is the maximum Vos (Vosmax), and
   when y>2, in any additional solutions between 1n and yn, the Vos is varied between Vosmin and Vosmax and, inversely, Vcs is varied between Vcsmin and Vcsmax;
   or, alternatively, Vos and Ncs are changed in each solution, and
   in the solution 1n, Vₒₛ is the minimum Vₒₛ (Vₒₛmin) and the N_{cs} is the maximum N_{cs} (N_{cs}max), and
   in the solution yn, N_{cs} is the minimum N_{cs} (N_{cs}min) and the Vₒₛ is the maximum Vₒₛ (Vₒₛmax), and
   when y>2, in any additional solutions between 1n and yn, the Vₒₛ is varied between Vₒₛmin and Vₒₛmax and, inversely, Nos is varied between N_{cs}min and Vₒₛmax,
2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.

In another preferred embodiment, the method of selecting the composition of a non-aqueous soaking solution further comprises the step of
1) preparing m series of 1n to yn individual soaking solutions by mixing os and cs wherein m is at least 1 and wherein in each of the m series
a) y is at least 2
b) cs is the same as in the first (benchmark) series, and
c) the os is changed and is different from the os of the first (benchmark) series, and
wherein in each individual solutions of each of the m series
d) Vₒₛ and Vcs are different in each solution, and
   in the solution 1n, Vos is the minimum Vos (Vosmin) and the Vos is the maximum Vcs (Vcsmax), and
   in the solution yn, Vcs is the minimum Vcs (Vcsmin) and the Vos is the maximum Vos (Vosmax), and
   when y>2, in any additional solutions between 1n and yn, the Vos varied between Vosmin and Vₒₛmax and, inversely, Vcs is varied between Vcsmin and Vcsmax;
   or, alternatively, Vos and Nos are different in each solution, and
      in the solution 1n, Vₒₛ is the minimum Vₒₛ (Vₒₛmin) and the N_{cs} is the maximum N_{cs} (N_{cs}max), and
      in the solution yn, N_{cs} is the minimum N_{cs} (N_{cs}min) and the Vₒₛ is the maximum Vₒₛ (Vₒₛmax), and
      when y>2, in any additional solutions between 1n and yn, the Vₒₛ varied between Vₒₛmin and Vₒₛmax and, inversely, N_{cs} is varied between N_{cs}min and N_{cs}max,
2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.

In a most preferred embodiment, the non-aqueous soaking Vcsmin and Vosmin are more than 0%.

A preferred volume of os is more than 5%, preferably at least 10%, or at least 15% or at least 20% more preferably between 5% to 25%.

A preferred volume of cs is preferably less than 95%, preferably less than 90%, or less than 85% or less than 80% more preferably between 75% to 95%.

In a most preferred embodiment, the Vcsmin or Ncsmin, Vcsmax or Ncsmax, Vosmin and/or Vosmax are the same in each of the m series and/or Vcs min or Ncsmin, Vosmax or Ncsmax, Vosmin and/or Vosmax are the same as in the first or benchmark series.

In another embodiment, each of the m series, the number of individual soaking solutions y is the same as the number of individual soaking solutions x of the first series; and/or the Vcs or Ncs is incrementally varied between Vcsmin or Ncsmin and Vcsmax or Ncsmax and, inversely, Vos is incrementally varied between Vosmin and Vosmax in any additional solutions between 1n and xn and/or in any additional solutions between 1n and yn.

In a preferred embodiment, in each of the m series, the number y of individual soaking solutions y is conserved and/or is the same as the number x individual soaking solutions of the first series and/or the Vcs or Ncs is incrementally varied between Vcsmin or Ncsmin and Vcsmax or Ncsmax and, inversely, Vos is incrementally varied between Vosmin and Vosmax in any additional solutions between 1n and xn and/or in any additional solutions between 1n and yn. In a further preferred embodiment, in any additional solutions between 1n and yn, the increment of Vcs or Ncs between Vosmin or Nosmin and Vcsmax or Ncsmax and of Vos between Vosmin and Vosmax is the same in the first series and in each of the m series.

In a preferred embodiment, the solutions of the m series are distributed in each compartment to form a second dimension. Typically, if the first dimension was represented by a line of wells (e.g. with the solution of the first series) in a standard screening or crystallization array, then the other lines of wells (e.g. containing the solutions of the m series) will generate columns of wells which will be the second dimension. Inversely, if the first dimension was represented by a column of wells (e.g. with the solution of the first series) in a standard screening or crystallization array, then the other columns of wells (e.g. containing the solutions of the m series) will generate lines of wells which will be the second dimension.

In another embodiment the crystals of a biological macromolecule are pre-conditioned in one or several suitable aqueous soaking solutions before transfer to the non-aqueous soaking solutions in order to, for example, equilibrate the crystals or equilibrate the crystals in several steps using solutions with stepwise reduced amounts of water for slow habituation of according crystals.

In a preferred embodiment, the biological macromolecule is a protein, preferably a binding protein, more preferably a receptor protein.

In another preferred embodiment the macromolecule is an RNA molecule. In a most preferred embodiment, the RNA molecule is a messenger RNA. More preferably, the mRNA comprises a riboswitch, more preferably the mRNA comprises an aptamer.

In a most preferred embodiment, the step of controlling the crystal is performed by standard methods of controlling the crystal. More preferably, step of controlling the crystal is performed by X-Ray-diffraction, neutron diffraction, absorption spectroscopy or reflectance spectroscopy in ultraviolet and visible spectral regions, infrared spectroscopy and/or visual analysis, most preferably by visual analysis followed by X-Ray analysis.

Generally and in the most preferred embodiment, the soaking solution is considered suitable for soaking the crystal when the quality of the crystal is sufficient to allow further uses and/or analysis on the crystal. Examples of such uses and/or analysis are electronic microscopy, neutron diffraction, small molecule screening, validation of hits from other screenings as, for example, hight-throughput (HTS) or biophysical screenings, reduction of protein disorder and biotechnological application of crystals as, for example, in chromatography, biosensors, material science (hybrid materials). Furthermore, crystals treated by this method can oftentimes be taken from their liquid environment and can be dried. This gives rise to much easier storage and transport. Furthermore, the diffraction quality often improves and is sustained over long periods of time. In a most preferred embodiment, a soaking solution is selected if it is suitable for a small molecule screening process.

In a particular embodiment of the present invention the biological macromolecular crystal is controlled at least once in a timeframe from 0 h to several weeks, preferably after 0 h, 1 h and 24 h, by inspection in particular by X-Ray in combination with preferably a visual inspection i.e. under the microscope.

In a particular embodiment of the present invention the specific soaking solution is controlled and subsequently selected for being suitable for soaking the crystal preferably by the use of in particular X-Ray and/or by visual control according to defined standard criteria commonly used by the expert in the relevant art of crystallography and in particular in X-Ray diffraction: highest resolution which should be less than 2.8 Å. preferably less than 2.5 A, low mosaicity, typically lower than 0.8°, no or minimal ice rings, high-quality electron densities as judged by an experienced crystallographer; and for visual control: no or minimal fractures, cracks, no or minimal dissolution, sharp crystal edges, colored in polarized light etc.) giving rise to signals in a subsequent analysis. In a further additional particular embodiment of the present invention, the step of controlling the crystal and selecting the suitable solutions is performed according to defined quality criteria such as resolution, mosaicity, electron density, presence of diffraction artifacts from, for example, unwanted ice crystals or salt crystals or other crystals grown from components of the ingredients of the soaking solution composition and crystal twinning).

In the present invention, the step of controlling the crystal can be performed by any of the method available to the skilled person to evaluate the presence and/or aspect of the crystal and suitability for further analysis such as X-ray-diffraction analysis. The step of controlling the crystal refers to assess the survival of biological macromolecular crystals in a particular soaking solution in terms of their presence and to measuring the impact of the soaking solution on the integrity/stability of the crystal. Preferred methods are X-Ray-diffraction, or neutron-diffraction performed at in house-sources or at synchrotons and visual analysis of the crystal by means of UV/VIS- and IR-spectroscopy.

In a preferred embodiment, the decision to select a soaking solution is made after visual control and/or after X-ray-evaluation. In a preferred embodiment, all kinds of crystals or crystal remains are subjected to control by X-ray-analysis since sometime crystals of bad appearance diffract nicely. In a preferred embodiment, only empty wells, where crystals dissolved or splintered completely are discarded.

The visual inspection is preferably to see how the appearance of crystals changes over time. In a preferred embodiment, the selection of soaking solution compositions is done after visual analysis of the crystal. Examples of non-suitable soaking solution compositions are those which resulted in complete dissolution of crystals or in severe damage like splintering are discarded. The first group of soaking solutions selected after visual control can then be subject to X-ray analysis. The skilled person sets up all control and selection parameters using no more than common general knowledge. The method of controlling the crystal and selecting the suitable soaking solution(s) according to the method of the present invention do not require any particular knowledge beyond common general knowledge of an expert in protein crystallization. The parameters of the controlling method such as the maximum resolution, low mosaicity, intact crystal structures, quality of electron density, missing twinning and absence of diffraction signals caused by undesired ice crystals will be set up to allow the standard control of the crystal integrity and the subsequent decision to select the suitable soaking solution.

In a preferred embodiment, the data collected in the controlling step of the crystal are analyzed to select the soaking solution. The soaking solution(s) which results improved the crystal's quality in terms of the quality criteria or the results of no or minimal detrimental impact on the crystal is/are selected. The selection criteria can be established by the crystallographer using common general knowledge.

In a preferred embodiment, the os and/or the cs contains small molecules as defined below and in particular small molecule fragments or molecular probes. In a preferred embodiment, the os contains small molecules as defined below and in particular small molecule fragments or molecular probes. In a preferred embodiment, small molecules or a cocktail of small molecules are used in a concentration typically ranging from micromolar to molar, preferably in a range from 5 mM to 500 mM, per individual soaking solution and even higher with saturation concentrations in OS or the total volume. The concentration of drug-size small molecule is around at least 5-20 mM. The concentration of fragment is preferably of at least of 10-50 mM. For screening purposes in both cases high concentrations (as long as without detrimental effects on the crystals) are desirable which can be up to 1M. In a preferred embodiment, the 100 mM of small molecules are dissolved in OS.

The present invention further concerns a method of selecting the composition of a non-aqueous soaking solution, wherein the os contains small molecules including molecule fragments and molecular probes to be analyzed, which diffuse into the biological macromolecular crystal.

Another embodiment according to the present invention is a method of selecting the composition of a non-aqueous soaking solution for soaking of biological macromolecules with and without small molecules to be analyzed and subsequent comparison with soakings of biological macromolecules with and without small molecules to be analyzed in aqueous soaking solutions in order to assess the influence of tightly bound water molecules for small molecule interactions with the biological macromolecule. In another preferred embodiment, the small molecules cannot be dissolved in water/aqueous environment.

When the soaking solution contains small molecules, a preferred embodiment consists in further performing a stress test on the crystal regarding the tolerance of a biological macromolecular crystal towards changes introduced by the addition of small molecules. Such stress conditions occur upon introduction of pH-changing small molecules or are caused by changes in osmotic pressure induced by high concentrations of small molecules. Therefore, the tolerance of a biological macromolecular crystal is tested towards pH changes by addition of hydrochloridic fragments and fragments and/or molecular probes at different concentrations to the formerly identified soaking conditions containing biological macromolecular crystals.

In another aspect of the invention, the method is a method of small molecule screening for a biological macromolecular crystal wherein the screening is performed in a soaking solution selected according to the method of the invention of selecting a soaking solution.

In another aspect, the invention is an array arranged to perform the above methods of the invention.

The invention also concerns the non-aqueous soaking solution obtainable by any of the above method of selecting the composition of a soaking solution. In a preferred embodiment, the soaking solution of the invention Vos is more than 5%, preferably, more than 10%, more than 20%, more than 30%, more than 40%, more than 50%, up to 90%.

The invention also concerns the use of the selected soaking solution for small molecule screening for a biological macromolecular crystal.

In a preferred embodiment where several solutions are tested, preferably simultaneously, more than one solution is selected thus defining a range of volume/percentage for each component of the tested solution.

In a further particular embodiment of the present invention a biological macromolecular crystal is incubated for 1 minute or less to 72 h, preferably 24 h, in the soaking solution containing the small molecules or a cocktail of small molecules.

In another particular embodiment of the present invention the biological macromolecular crystal is vitrificated at cryogenic temperatures or alternatively subjected to a direct X-ray measurement at room temperature.

In an additional particular embodiment of the present invention the biological macromolecular crystal is stored at cryogenic temperatures in preferably liquid nitrogen and or helium or said biological macromolecular crystal is directly subjected to data collection by imaging techniques comprising X-ray, electron and neutron diffraction.

In the present invention individual soaking solution compositions suitable for a small molecule screening process of said biological macromolecular crystal means that soaking solution is judged as “suitable” in particular for a small molecule screening process if it allows to perform a “Hit picking” process wherein the binding of a given small molecule to the biological macromolecular crystal is defined as “hit”.

In the present invention individual soaking solution compositions suitable for a small molecule screening process of said biological macromolecular crystal means that soaking solutions are judged as “suitable” if it enables the soaking of fragments and small molecule ligands into a biological macromolecular crystal resulting in highest resolution possible in diffraction experiments less than 2.8 Å preferably less than 2.5 Å in case of molecular probes and fragments and to less than 3.5 Å in case of drug-size molecules. Ideally, the biological macromolecular crystal shows minimal mosaicity, typically less than 0.8°; reveals an electron density which is then evaluated i.e. by an experienced crystallographer.

Subject matter of the present invention is a method according to the above described invention, wherein said method is automated.

In a further embodiment, the subject matter of the invention concerns the soaking solution selected by any of the above embodiment of the method of selecting the composition of a soaking solution of the invention. In a preferred embodiment, the soaking solution comprises at least 5% of os.

In a further embodiment, the subject matter of the present invention is the use of the soaking solution selected by any of the above embodiment of the method of selecting the composition of a soaking solution for small molecule screening for a biological macromolecular crystal.

In a further embodiment, the subject matter of the invention is an array arranged to perform the method of the invention and an array containing the solution as prepared using the method of the invention, in particular the preparation step 1 of the above methods, or an array containing the soaking solution(s) selected by the method of the invention of selecting the composition of soaking solutions. A preferred embodiment according to the present invention is an array arranged to perform the method as described previously, wherein said array comprises a first dimension of at least two individual non-aqueous soaking solutions (1n to xn) and a second dimension of at least two individual non-aqueous soaking solutions (1m to ym) wherein each of said soaking solutions is located in a separated compartment of said array, and wherein each of said soaking solution comprises an organic solvent (os) and a compatible solute (cs).

Another preferred embodiment according to the present invention is an array as described previously, wherein the array comprises of 1x to xn series in said first dimension of individual soaking solutions for the biological macromolecule wherein the compatible solute of each of the series of individual non-aqueous soaking solution in the first dimension differs from the one of the series of individual soaking solutions of the second dimension and the organic solvent of each of the series of individual non-aqueous soaking solution in the first dimension differs from the one of the series of individual soaking solutions of the second dimension.

### Definitions

### Definition of Compartment

A compartment is a container and refers to a separate section or part in the array used in particular for culturing or screening. In the present invention a compartment can be a single well of an array or equally can refer to any container or section of an array or part in which experiments can be performed. This includes any types of wells from commercially available and custom made (i.e. printed in a 3D printer, etc.) plates of any suitable materials and any other devices that can be used for soaking and/or screening. The term “compartment” can also refer to parts or organelles of living or preserved cells which comprise but are not limited to mammalian, insect and bacterial cells or additionally to (bio-) chips, foils, tubular or diverse shaped tubes made from any suitable materials.

### Definition of Array

An “array” is one or a series of compartments or containers or comprising an arrangement of elements (i.e. crystals) in a pattern on a solid support and/or vessels. In the present invention, an array can also mean one single container/compartment. When the array is of one single compartment it can also refer to a tube or a well. Although the pattern of an array is arranged typically in a two dimensional space, in particular, a standard screening array or commercial array for high throughput screening composed of a multitude of individual compartments or wells forming lines on 2 different perpendicular axis. The pattern may also be arranged in a one (e.g. a tube or a single well or a single line of wells) or three dimensional space (e.g. the superposition of at least 2 arrays having two dimensions. However the present invention is not limited to a straightforward spatial organization. As an example, information technology systems can be used to generate logical arrays, in which the distribution, the specification and the location of the respective components in the array can be tracked (i.e. in a table/sheet), but are not characterized by a straightforward spatial organisation. Furthermore, the term array can also refer to different formats, which include “chips” or “biochips.” Here, a range of low density defined as i.e. two to ten different addressable locations to high density i.e. one thousand or more addressable locations can be arranged in or on the array. It shall be mentioned that the shape and size of the array chip or biochip can be irregular and is not limited by any geometrical format, even though the typical chip array format is rectangular. The configuration of the array can comprise a bundling, mixing and or homogenous blending of a plurality of addressable locations, which are suitable for i.e. high-throughput handling and robotic delivery of reagents. The spatial organisation of the arrays furthermore allows the detection and or quantification of a given signal and includes but is not limited to chemical luminescence, X-ray, confocal or deflective light gathering and laser illumination. Array formats comprise any appropriate format for culturing and soaking biological macromolecular crystals including but not limited to microarrays, microchips, arrays of biomolecules attached to multiwall plates and living organisms such as insect, bacterial and mammalian cells (“in vivo crystallization”). Further examples for an array format comprise serial crystallography, foils and tubular shaped devices on or in which crystals can be subjected to X-Ray measurements.

### Definition of Series of Individual Soaking Solutions

A series of individual soaking solutions refers to at least two individual soaking solutions which have several features in common. In particular the individual soaking solutions of series may preferably have at least one of their components in common e.g. the same organic solvent (os), a compatible solute (cs). The common features can be the same volume of 1, 2, 3 or more of the components, the ratio of the volume of some of the components to 1 or more components and/or the number of individual solutions.

When more than one series is prepared, one is called “first series” but it can also equally be named “benchmark series”. This series is conveniently called “first” as it is often distributed in the first line of an array. However, the so called first can equally be named a “reference” or “benchmark” series and is not necessarily distributed in the first line or column of wells of the array. Actually, this benchmark series is used as a reference for the preparation of the additional series according to the method of the invention. When more than one series is prepared, the so called first series or benchmark series can therefore be any one of the series which will then be used as benchmark for the rule-based method of the invention.

In a preferred embodiment, a series of at least two individual soaking solutions refers to the composition of at least two soaking solutions, which comprises the volumes of an organic solvent, a compatible solute, wherein in each series different compatible solutes are used.

### Definition of Protein, Polypeptide and RNA

Polypeptide: The term “polypeptide” refers to any oligomeric arrangement of amino acids, which are typically but not exclusively covalently joined by peptide bonds and occur at variable lengths. In general, the term “polypeptide” refers to an amino acid sequence with less than 70 amino acids and a molecular weight of less than 70 kDa. The source of the polypeptide comprises a natural or unnatural origin, or a combination thereof including a naturally occurring polypeptide, a polypeptide produced by recombinant molecular genetic techniques, a polypeptide from a cell or translation system, or a polypeptide produced by cell-free synthetic means. The sequence of the amino acids in a polypeptide determines its structure and is not limited to full-length sequences, but can be partial or complete sequences. In general, the term “peptide′ refers to a small polypeptide which consists of 2-25 amino acids in length, whereas a native polypeptide relates to a polypeptide having a sequence of amino acid residues which is identical to the one found in nature (e.g. the wild-type polypeptide). Moreover, a native polypeptide can have a natural origin and be isolated from its naturally occurring source (i.e. a plant cell or tissue) or be genetically engineered using recombinant genetic techniques. Posttranslational modifications may or may not occur in native polypeptides depending on the naturally occurring wild-type polypeptides. The term polypeptide can also refer to a polypeptide fragment. As used herein, this term refers to any contiguous subset of the full-length polypeptide amino acid sequence. A polypeptide fragment or portion can be isolated from any domain of the polypeptide and is not limited to a specific length ranging from about 4 amino acids to up to a full-length polypeptide sequence. Furthermore, a polypeptide can possess or not possess any given biological or synthetic activity. The term “protein′ refers to a polymer of amino acid which are linked together by peptide bonds. The term, as used herein, refers to proteins, polypeptides, and peptides of any size, structure, or function. As laid out in the definition of a polypeptide, a protein may have a natural origin, recombinant (i.e. biotechnologically produced in genetically modified organisms or transiently transfected cell cultures, with or without mutations, etc.), synthetic or comprise any combination of the above mentioned. Furthermore, the term “protein” refers to single molecules or to complexes of multiple molecules (complexes of proteins with i.e. other peptides, polypeptides and proteins). The term protein may also apply to amino acid polymers containing one or more amino acid residues of the artificial chemical analogue of a corresponding naturally occurring amino acid. Furthermore the term protein may also refer to prions which are misfolded proteins with the ability to transmit their abnormal shape onto harmless variants of the same protein.

RNA is a polymeric biomolecule consisting of ribose nucleotides (nitrogenous bases appended to a ribose sugar and attached by phosphodiester bonds) that forms strands of varying lengths and structural forms and conformations (for example: bulges and helices, circles etc.). The nitrogenous bases include adenine, guanine, cytosine and uracil. RNAs can be broadly classified based on the lengths of the chain of nucleotides and comprise small RNAs (200 or fewer nucleotides) and long or large RNAs (200 or more nucleotides). The group of small RNAs comprises but is not limited to transfer RNA (tRNA), 5S ribosomal RNA (rRNA), microRNA (miRNA), small interfering RNA (siRNA), small nucleolar RNA (snoRNA), Piwi-interacting RNA (piRNA), tRNA-derived small RNA (tsRNA), and small rDNA-derived RNA (srRNA), whereas the group of long or large RNAs comprises but is not limited to long non-coding RNA (lncRNA) and messenger RNA (mRNA in particular mRNA comprising a riboswitch (see definition in the next paragraph). However, the term RNA may also refer to the genetic material (2 to 20 kb) of RNA viruses comprising double stranded RNA, single-stranded RNA(+/−), isometric particles with single-stranded RNA, pseudoviridae (i.e. Ebola, SARS, influenza, etc.). Also, the term RNA may refer to viroids or virusoide/satellite viruses. Viroids are single-stranded, covalently closed circular RNA molecules characterized by a small size (250-400 nucleotides), that do not encode for any proteins and lack a capsid (but contain a protein coat), whereas virusoides or satellite viruses refer also to circular subviral particles with single-stranded RNAs, but are dependent on viruses for replication and encapsidation. The genome of virusoide usually consists of several hundred nucleotides (200-400).

Riboswitches are regulatory segment of a messenger RNA molecule that binds a small molecule, resulting in a change in production of the proteins encoded by the mRNA and are often conceptually divided into two parts: an aptamer and an expression platform. The aptamer directly binds the small molecule, and the expression platform undergoes structural changes in response to the changes in the aptamer. The expression platform is what regulates gene expression. A preferred embodiment of the present invention is wherein the biological macromolecule is from mRNA comprising the aptamer of a riboswitch.

### Definition of Binding Site

A macromolecular binding site refers to any position on a given macromolecule (such as a protein or mRNA etc.) that binds with specificity to another molecule. This binding partner is called i.e. a ligand and may include other proteins resulting in a protein-protein interaction or small molecules comprising i.e. enzyme substrates, hormones, allosteric modulators, second messengers, and inhibitors. Binding events may result in conformational changes of the respective macromolecule resulting in an alteration of its function or activity like in proteins and riboswitches in mRNA for instance. Most of such binding events are non-covalent reversible (transient), less often also covalent reversible or irreversible. Types of binding sites include active sites (orthosteric sites) and allosteric sites.

Active sites bind substrates to induce a chemical reaction as well as inhibitors that inhibit the respective reaction. Allosteric sites are regulatory sites that bind ligands that may enhance or inhibit a macromolecules function.

### Definition of Soaking Solution

A soaking solution is any solution that serves as liquid environment of a biological macromolecular crystal that differs in its composition from the original environment in which the biological macromolecular crystal nucleated and grew. Usually it is derived from the original crystal growth environment which contains additionally organic solvents or compatible solutes and other components. The component of the soaking solution that is derived from the original growth environment may be of the same concentration of its individual components or of multiples of this concentration or diluted.

In the present description of the invention, the term non-aqueous and water free are used. Both terms refers to the absence of water and have the same equivalent meaning.

Soaking solutions are usually applied to introduce either small molecules as molecular probes, fragments and drug-size molecules to the crystal in order to elucidate binding events of respective small molecules in subsequent diffraction experiments. Furthermore, soaking solutions are applied to introduce heavy-atoms to the biological macromolecular crystals in order to obtain the phases for solving hitherto unknown structures in the course of subsequent diffraction experiments. Furthermore, they are applied to introduce cryoprotectants to mediate cryo-stress resistance, to prepare crystals for biotechnological applications (for example chromatographic applications or biosensors) and/or to improve crystal quality. The volume of a soaking solution ranges usually from μm to ml, typically between 1 to 10 μl. In case of automation volumes in the scale of nanoliters can be adjusted accordingly.

The volume of the individual soaking solutions ranges between pico- to milliliter.

Unless the volume is expressed specifically in standard measures of volume e.g. μm, ml, “V” such as in Vos, Vcs always refers to the proportion of the relevant component of the solution to the total volume (VT) of the solution i.e. 100%. For instance Vos refers to the percentage/proportion of organic solvent (os) in the whole volume of solution e.g. Vos=10 means os is 10% of the total volume of the solution while Vos=10 μl means 10 μl of os solution.

A preferred volume of os is more than 5%, preferably at least 10%, or at least 15% or at least 20% more preferably between 5% to 25%.

A preferred volume of cs is preferably less than 95%, preferably less than 90%, or less than 85% or less than 80% more preferably between 75% to 95%.

“N” such as in Nos, Nos, Nors and Nw always refers to the number of moles of the relevant component. NT refers to the total number of moles of the components in the solution, ie. NT=Ncrs+Nw+Nos+Ncs.

In a preferred embodiment, the ratio of the number of moles of compatible solute to the total number of moles of the components (Ncs/NT) is less than 0.95, more preferably less than 0.9, or less than 0.85, or less than 0.8, more preferably between 0.75 and 0.95.

### Definition of Small Molecule Especially Molecular Probe, Fragment and Drug-Size Small Molecule

In the field of molecular biology and pharmacology the term “small molecule” refers to any low molecular weight organic compound with a molecular weight usually in the range of 100-500 Da, but can extend in special cases up to about 2000 Da (e.g. in case of macrolides or proteolysis targeting chimeras (PROTACs), in contrast to high molecular weight compounds like polymers as RNA, DNA, proteins or polysaccharides. Small molecules even comprise monomer or oligomer constituents of RNA, DNA, proteins (peptides) and polysaccharides as there are ribo- and deoxyribonucleotides, amino acids, mono- or oligosaccharides. Many small molecules regulate biological processes or are otherwise relevant in biological systems. As such, they can exert their effect for example as agonist and antagonist, as inhibitor or mediator of protein-protein interfaces, proximity or interaction, as regulator of biophysical properties like osmotic pressure etc. They can have a variety of functions in for example (but not limited to) signal transduction, metabolism, regulation of osmotic pressure, cryoprotection. They can have applications for example as drugs, molecular probes, starting points for drug discovery, serve as lead compounds, clinical candidates, as pesticides, cosmetics, food chemistry, fragrances, fungicide and many others. Their origin can be natural as secondary metabolites or synthetic as drugs and can have beneficial effects as therapeutics or detrimental effects like poisons. In drug discovery campaigns small molecules are used to screen for interactions with target macromolecules and may have or may not have binding affinity to respective targets or may or may not alter biological processes. They can be used as molecular probes or as small molecule fragments.

A “molecular probe” is a small molecule of 30-120 Da with an attached functional group (e.g. amino group, acid group, hydroxyl group etc.) used in molecular sciences such as biochemistry, structural biology or chemistry or medicinal chemistry to study the properties of other molecules especially biologically relevant macromolecules (RNA, DNA, proteins, peptides, polysaccharides) and to explore their interaction behavior. Therefore, a molecular probe can be a group of atoms or molecules used in molecular biology or chemistry to study the properties of other molecules or structures. If some measurable property of the molecular probe used changes when it interacts with the analyte (such as a change in absorbance), the interactions between the probe and the analyte can be studied. Radioactive DNA or RNA sequences are used in molecular genetics to detect the presence of a complementary sequence by molecular hybridization. In computational approaches molecular probes can even be artificial constructs that reflect predominantly one property, e.g. of a hydrogen-bond donor or a lipophilic probe. If some measurable property of the applied molecular probe changes when it interacts with the analyte (such as a change in absorbance or the interaction geometry), the interactions between the probe and the analyte can be studied. In molecular sciences such as structural biology (e.g. crystallography) molecular probes are used to map properties of binding sites of macromolecular targets as there are: hydrophobic, hydrophilic properties H-bonding patterns, induced polarization effect resulting shifts of protonation states, conformational changes and induced molecular adaptations. Furthermore, molecular probes provide information about putative pharmacopohore patterns, which help to define the requirements which set of small molecules is applied in screenings. Furthermore, a molecular probe can be used to perform stress tests for example to assess a macromolecule's or a macromolecular crystal's tolerance towards for example organic solvents or pH changes. Molecular probes enable to indirectly study the properties of compounds and structures, which may be hard to study directly. The choice of molecular probe will depend on which compound or structure is being studied as well as on what property is of interest.

The term “small molecule fragment,” refers to a small molecule (e.g. ≤250 kDa in size) typically having a KD ranging usually from low one digit-micromolar up to two-digit millimolar affinity. A small molecule fragment is typically 120-250 Da in size and serves as starting points for optimization by chemical synthesis for example to create ligands such as inhibitors, agonists or antagonists. The small molecule fragment can be, for example, a peptide, any organic molecule or natural product, a short nucleic acid sequence (e.g., DNA, or RNA type), and/or any other ligand including solvent molecules and cryo-buffer ingredients capable of binding to the macromolecular target. The “target” can be any macromolecular system with a biological function or macromolecular crystal system based on amino acids, nucleic acids as monomeric building blocks and additional sugar or lipid moieties; for example, enzymes, soluble proteins or membrane-bound proteins such as soluble or membrane-bound receptors or ion channels, RNA and DNA molecules or sugar-based macromolecules.

“Drug-size molecules” are organic compounds of 250-2000 Da, that may or may not exert a therapeutic effect as drug or lead candidate. Exerting such an therapeutic effect they serve the modulation of some kind of biological activity like a macromolecule's activity for example as agonist or antagonist or as catalyst like in targeted protein degradation. Drug-size molecules comprise, but are not limited to, drugs, agrochemicals, aromatic substances or fragrances.

One embodiment of the method of the present invention is to test a collection of small molecules in a crystal system of a biological macromolecule in order to separate small molecules which interact with the respective biological macromolecule from the small molecules of the collection that do not interact with respective biological macromolecule.

Typically, in the array and method of the invention, the small molecules are dissolved in the os.

### Definition of Organic Solvent (os)

A solvent is a substance capable of dissolving other substances. The term organic solvent (os) refers to any carbon-based solvent, protic or aprotic, of low reactivity that is capable of dissolving or dispersing one or more other substances. Carbon-based means that these solvents at least contain one carbon atom. Furthermore, they contain most often at least one hydrogen atom. The terms “liquid carbohydrates” and “carbon-based solvents” have the same meaning and are used interchangeably.

Examples of suitable organic solvent are a straight or branched chain monohydric aliphatic alcohol containing from 1 to 7 carbon atoms, such as ethyl alcohol or isopropyl alcohol; a dihydric aliphatic alcohol containing from 2 to 7 carbon atoms, such as hexylene glycol; a monoalkyl ether of an aliphatic dihydric alcohol containing a total of 3 to 6 carbon atoms, such as the monomethyl, -ethyl and -butyl ethers of ethylene glycol; or a dialkyl ketone containing a total of 3 to 5 carbon atoms, such as acetone. Other solvents that can be used are benzyl alcohol or phenyl ethyl alcohol. Preferred solvents are ethyl alcohol, hexylene glycol, the monomethyl ether of ethylene glycol, and acetone. Mixtures of solvents can also be used.

Organic solvents may be selected from the preferred group comprising but not limited to: methylsulfinylmethane (dimethyl sulfoxide (DMSO)) and derivatives, especially phospoderivatives, ethyl acetate and derivatives, methanol, ethanol, propan-1-ol, propan-2-ol, N,N-dimethylformamide, oxolane (tetrahydrofuran), ethoxyethane (diethyl ether), crown ethers, 1,4-dioxane (dioxane), furan and others suitable to dissolve small molecules for protein crystal-based screenings.

According to the present invention, mixtures of single organic solvents selected from the above mentioned list of preferred organic solvents can be used. Thus, the term organic solvents according to the present invention may refer to a mixture of organic solvents. According to the present invention, mixtures of single organic solvents selected from the above mentioned list of preferred organic solvents can be used in the array.

In a preferred embodiment, small molecules are dissolved in the os.

### Definition of Compatible Solute (Osmoprotectant, Osmolyte or Cryoprotectant)

Compatible solute (cs), osmoprotectants or osmolytes comprise organic compounds of natural or synthetic origin that prevent proteins from precipitation or denaturation under stress conditions and keep polypeptides, proteins and RNA regarding their spatial organization/conformation stable and soluble. Such stress conditions includes elevated or lower temperature changes (including cryo-conditions), changes in osmotic stress, presence of protein denaturing compounds like urea, drought, radiation, radicals, poisons as well as pressure. Natural compatible solutes are of high solubility in water and of low toxicity towards organisms even at high concentrations thereby acting as osmolytes and help organisms survive extreme osmotic stress. Compatible solutes may be selected from the group consisting of methylamines, polyols (including polymers, mono-, di-, trisaccharides, cyclitols), amino acids and others.

Compatible solutes have been shown to stabilize macromolecule conformation and in particular to induce the adoption of distinct conformations in proteins and RNA molecules.

In a preferred embodiment, the compatible solute is selected from the group consisting of methylamines, polyols including polymers, mono-, di-, trisaccharides, cyclitols, and amino acids. In a most preferred embodiment, the cs is poly(oxyethylene) of a molecular weight of 400 Da (PEG400) or 3350 Da (PEG3350), 2-Methylpentane-2,4-diol (MPD), ethane-1,2-diol (ethylene glycol, EG) and/or propane-1,2,3-triol (glycerol).

In some embodiment, the cs is liquid and is added to the soaking solution at the volume Vos. Examples of liquid cs are poly(oxyethylene) of a molecular weight of 400 Da (PEG400), propane-1,2,3-triol (glycerol), ethane-1,2-diol (ethylene glycol), 2-Methylpentane-2,4-diol (MPD).

In some embodiment, the cs is solid and is added to the soaking solution dissolved in organic solvents whereby Vos refers to the volume of the respective cs dissolved in os. Examples of solid cs are N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-aminoethanesulfonic acid (taurine), 2-aminoethanesulfinic acid (hypotaurine), (2S)-pyrrolidine-2-carboxylic acid (proline).

In an additional particular embodiment of the invention the preferred group of methylamines comprises N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine) as well as respective possible stereoisomers and derivatives.

In a further additional particular embodiment of the invention the preferred group of amino acids comprises 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), ß-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[(1R)-1-carboxyethyl]amino]-5-(diaminomethylideneamino)pentanoic acid (octopin) as well as respective stereoisomers and derivatives.

In an additional particular embodiment of the invention the preferred group of polyols comprises poly(oxyethylene), polyhydric alcohols, monosaccharide, disaccharide, trisaccharide, cyclitols and derivatives of former molecules.

In an additional particular embodiment of the invention the preferred group of polyols comprises poly(oxyethylene) of diverse molecular weights (for example polyethylene glycol 200, polyethylene glycol 400, polyethylene glycol 600, polyethylene 3350 (also referred as 3350 or macrogol 3350)), and polyhydric alcohols (propane-1,2,3-triol (glycerol), ethane-1,2-diol (ethylene glycol), 2-Methylpentane-2,4-diol (2-Methylpentane-2,4-diol, MPD), (3R,4S,5S,6R)-2-(2,3-dihydroxypropoxy)-6-(hydroxymethyl)oxane-3,4,5-triol (1-glucosylglycerol)). In a preferred embodiment, polyethylene glycols are of molecular weights ranging between 200-20000 g/mol and includes but is not limited to poly(oxyethylene) (200 g/mol, 300 g/mol, 400 g/mol, 550 g/mol, 600 g/mol, 1000 g/mol, 1500 g/mol, 2000 g/mol, 3350 g/mol, 4000 g/mol, 5000 g/mol, 6000 g/mol, 8000 g/mol, 10000 g/mol, 20000 g/mol).

In a preferred embodiment, the compatible solute is a mix of at least 2 poly(oxyethylene)s preferably polyethylene glycol 400 and 3350.

In an additional particular embodiment of the invention the preferred group of monosaccharides comprises (preferably: (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside).

In an additional particular embodiment of the invention the preferred group of disaccharides comprises (β-D-Fructofuranosyl α-D-glucopyranoside (sucrose), (2R,3S,4S,5R,6R)-2-(Hydroxymethyl)-6-[(2R,3R,4S,5S,6R)-3,4,5-trihydroxy-6-(hydroxymethyl)oxan-2-yl]oxyoxane-3,4,5-triol (trehalose)).

In an additional particular embodiment of the invention the preferred group of trisaccharides comprises (preferably (2R,3R,4S,5S,6R)-2-[(2S,3S,4S,5R)-3,4-dihydroxy-2,5-bis(hydroxymethyl)oxolan-2-yl]oxy-6-[(2S,3R,4S,5R,6R)-3,4,5-trihydroxy-6-(hydroxymethyl)oxan-2-yl]oxymethyl]oxane-3,4,5-triol (raffinose)), sugar alcohols (preferably (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol), (2R,3S,4R,5S)-hexane-1,2,3,4,5,6-hexol (galactitol)).

In an additional particular embodiment of the invention the preferred group of cyclitols comprises (preferably (1S,2S,4S,5R)-6-methoxycyclohexane-1,2,3,4,5-pentol (pinitol)).

In an additional particular embodiment of the invention the preferred group of derivatives of former molecules comprises preferably mannose derivatives as fiorin ((2R)-O2-(β-D-mannopyranosyl)glyceric acid and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose as well as respective stereoisomers and derivatives.

In another particular embodiment of the invention the preferred group of other molecules comprises u derivatives, 1-methyl-2-pyridinecarboxylic acid (homarine), methylsulfonium solutes as 3-dimethylsulfoniopropanoate (Dimethylsulfoniopropionate), and methylsulfinylmethane as dimethyl sulfoxide and other compounds with traits of compatible solutes according to definition as well as respective stereoisomers and derivatives.

### Definition of Crystallization Solution (crs)

The crystallization solution (crs) refers to any solutions used for the preparation of a crystal at any point in time of the course of any standard crystallization process, in particular for nucleation, growth and/or storage of the crystal in conditions suitable to preserve the integrity of the crystal for any further uses. As a way of examples, crs of the invention can be defined under various terms known in the art such as mother liquor, medium of nucelation, medium of crystal growth, reservoir solution, storage solution. In particular, crystallization solutions comprise the mother liquor and other solutions like reservoir solutions at any state in the process of crystallization.

In a preferred embodiment, the crystallization solution is obtained from the crystallization process of the biological macromolecular crystal and typically comprises pH-maintaining buffer substances, precipitants and additives. In the present invention, crs refers to any solution that is used for the preparation of the crystallization of macromolecules using any standard method of crystallization known in the art. Ducruix, A. & Giege, R. (eds.) Crystallization of Nucleic Acids and Proteins (Oxford University Press, Oxford, 1999); 1. Gavira, J. A. Current trends in protein crystallization. Archives of Biochemistry and Biophysics 602, 3-11 (2016). 1. McPherson, A. & Gavira, J. A. Introduction to protein crystallization. Acta Crystallographica Section F Structural Biology Communications F70, 2-20 (2014).

The mother liquor is any solution containing concentrated proteins, polypeptides or RNAs for crystallization. In addition, it might contain pH-maintaining buffer substances, precipitants and additives. A reservoir solution is any solution that contains pH-maintaining buffer substances precipitants and additives but usually no protein that in respect to some vapor-diffusion techniques serves to drive the proteins in the mother liquor into supersaturation. Supersaturation is a non-equilibrium condition in which some quantity of the proteins, polypeptides or RNA is present in excess of the solubility limit. Equilibrium is re-established in the process of crystallization. Supersaturated solutions are produced by modification of properties of undersaturated solutions in order to reduce the ability of the medium to solubilize the macromolecule. Techniques for achieving supersaturation comprise bulk crystallization, batch method in vials, microbatch under oil, controlled evaporation, bulk dialysis, concentration dialysis, microdialysis, free-interface diffusion, counter-diffusion in capillaries, liquid bridge, vapor diffusion on plates (sitting drop), vapor diffusion in hanging drops, sequential extraction, pH-induced crystallization, temperature-induced crystallization, crystallization by effector addition, in vivo crystallization. Since the compositions of mother liquor and reservoir solution are altered in the process of the afore mentioned techniques (for example by dialysis or vapor diffusion) the term mother liquor and reservoir solution, compiled in the term crystallization solution, apply to respective solutions at any point in time of the course of the crystallization process.

### Method for Controlling the Biological Macromolecular Crystal and Selection of the Suitable Soaking Solution

In the present invention, the step of controlling the crystal can be performed by any of the method available to the skilled person to evaluate the presence and/or aspect of the crystal and suitability for further analysis such as X-ray-diffraction analysis. The step of controlling the crystal refers to assess the survival of biological macromolecular crystals in a particular soaking solution in terms of their presence and to measuring the impact of the soaking solution on the integrity/stability of the crystal. Preferred methods are X-Ray-diffraction, or neutron-diffraction performed at in house-sources or at synchrotons and visual analysis of the crystal by means of UV/VIS- and IR-spectroscopy.

In particular embodiment of the present invention the crystal is controlled and the soaking solution is subsequently selected by the use of routine technics such as standard X-Ray analysis and/or by visual control under a microscope. In a preferred embodiment, visual control will be followed by X-ray analysis.

In a fully automated setup, a robot would grap the experimental plate, an Artificial intelligence would identify crystals in a well (or discard the according well if the crystals are dissolved or in a bad condition) and the chosen crystals are transferred automatically to an X-Ray beam. Preferably, the ultimate decision is made by X-Ray-evaluation.

Also, the visual inspection can serve to identify crystals in the well of an experimental plate that are worthwhile to subject to X-Ray-analysis. In a preferred embodiment, all kinds of crystals or crystal remains are subjected to control by X-Ray-analysis since sometime crystals of bad appearance diffract nicely. In a preferred embodiment, only empty wells, where crystals dissolved or splintered completely are discarded.

Visual inspection can be done under a microscope or using UV/VIS- or IR-spectroscopy to identify the surviving crystals that can be subjected to X-Ray-analysis.

The visual inspection is preferably to see how the appearance of crystals changes over time. This can be useful to estimate the maximum soaking time.

The visual data can be collected in order to correlate osmotic conditions in the array with cracking behavior etc.

In a preferred embodiment, a first selection of soaking solution compositions can be done after visual analysis of the crystal. Soaking solution compositions that resulted in complete dissolution of crystals or in severe damage like splintering are discarded. The first group of soaking solutions selected after visual control can then be subject to X-ray analysis. This concerns crystals from soaking solution compositions that, under visual analysis show no signs of damage (that is sharp edges and smooth faces, no cracks, visible color under polarized light) and signs of moderate damage that does not compromise the general possibility to transfer and subject the according crystals to x-ray or neutron-diffraction experiments. With the X-ray- or neutron-diffraction analysis, the skilled person sets up all parameters using no more than common general knowledge.

The method of controlling the crystal and selecting the suitable soaking solution(s) according to the method of the present invention do not require any particular knowledge beyond common general knowledge of an expert in protein crystallization. The parameters of the controlling method such as the maximum resolution, low mosaicity, intact crystal structures, quality of electron density, missing twinning and absence of diffraction signals caused by undesired ice crystals will be set up to allow the standard control of the crystal integrity and the subsequent decision to select the suitable soaking solution.

For visual and X ray controls, the parameter can be conveniently reported in a template like in FIGS. 5-6 and 8-9.

For visual inspection/control, the observer notes the presence or absence of minimal fractures, cracks, if no or minimal dissolution, sharp crystal edges will be evaluated, color s in polarized light etc. The selection criteria, for visual inspection in particular, can be based on the presence or absence of cracks or on the type of cracks in the crystal. Typically, the types of cracks can be classified (longitudinal, lateral, crisscross cracks). The appearance of the crystals surface (e.g. smooth, rough) or edges (e.g. sharp, roundish) and their behavior in polarized light (strong color, some color, no color) are standard criteria used to measure the impact of the soaking solution composition on the crystal and to select the soaking solution(s) which best preserve the integrity of the crystal. In the most cases not all of the crystals survive in all the conditions. Some crystals completely dissolve over time or are damaged while the integrity of other crystals is fully conserved.

The X-Ray examination can take place with commercially available x-ray source as well as at a synchrotron. Commercially available x-ray sources are much slower in data collection. For that reason, at least two pictures at different crystal orientations, typically 0° and 90, are taken in order to safe time. These images are a sufficing basis for an evaluation of the crystals quality after subjecting them to according soaking solutions in terms of resolution, and mosaicity, the appearance of artifact like data originating from unwanted ice crystals in the protein crystal and sometimes a well-known phenomenon called “twinning”.

For the x-ray examination the crystals are preferably removed from the cryo-vials or any other storage containers and mounted on an x-ray or neutron diffraction machine for instance in a so-called cryo-stream that maintains the cryogenic temperature around the protein crystal. In case of x-ray studies at room temperature, no cryo-stream is required. The skilled person will typically define the standard criteria commonly used by the expert in the art and in particular highest resolution which should be less than 2.8 Å. preferably 2.5 Å, low mosaicity, typically lower than 0.8°, no or minimal ice rings, high-quality electron.

The data collected in the controlling step of the crystal are analyzed to select the soaking solution. The soaking solution which results improved the crystal's quality in terms of the quality criteria or the results of no or minimal detrimental impact on the crystal are selected. The selection criteria can be established by the crystallographer using common general knowledge.

Data collection at a synchrotron is much faster. In this case, complete data sets are collected and evaluated since the appearance of the so called “electron density” becomes an additional criterium of validation judged by an experienced crystallographer as suitable or not suitable.

In a particular embodiment of the present invention the biological macromolecular crystal is controlled at least once in a timeframe from Oh to several weeks, preferably after 0 h, 1 h and 24 h, by inspection in particular by x-ray in combination with preferably a visual inspection. The repeated inspection over time serves to determine the maximal amount of time for soaking experiments that maintain suitable diffraction quality in diffraction experiments.

Based on the data collected by X-Ray, neutron diffraction and/or visual analysis a decision is made on which soaking solutions is selected for further application in particular for small molecule fragment screening, for molecular probe analysis, screening for more drug-size small molecules, to validate HTS-hits or hits from biophysical screenings.

In contrast to traditional optimizations of soaking solutions which result in one single soaking condition, the method of the invention usually results in a spectrum of soaking conditions. From this spectrum the experimenter can further select within the selected spectrum the very best soaking solution to address any detrimental effects on the crystal that may be caused by for example respective utilized small molecule fragments or pH changes resulting from their utilization etc.

In a preferred embodiment, a further method of control which consists in performing stress tests on the crystals can be used. The method is particularly used to evaluate the tolerance of a biological macromolecular crystal to changes introduced by the addition of small molecules.

Typically, the stress test will be performed as a further step following any of the above mentioned method of controlling the crystal selection. The stress tests can typically be used to fine-tune the selection of a first spectrum of soaking solutions first controlled and selected using X-Ray, or neutron diffraction, performed at inhouse sources or at synchrotons and visual analysis of the crystal.

For the stress tests, the volume and components of the selected solutions are conserved but the os contains small molecules called stress probes. Such stress conditions occur upon introduction of these small molecules which results in pH-changes or in changes in osmotic conditions using high concentrations of small molecules. The stress test typically consists in the addition of for example hydrochloridic fragments/probes for stress tests and fragments dissolved at different concentrations in the organic solvent of the formerly selected soaking solutions.

The following embodiments are part of the present invention:
1. Use of a non-aqueous soaking solution for soaking the crystal form of a biological macromolecule, for testing the integrity of a biological macromolecular crystal or for a screening process of a biological macromolecular crystal or for identifying a specific soaking solution composition that is suitable for a screening process of said biological macromolecular crystal, wherein the non-aqueous soaking solution comprises at least an organic solvent (os) and at least a compatible solute (cs).
2. Method of selecting the composition of a non-aqueous soaking solution suitable for soaking the crystal form of a biological macromolecule wherein the solution comprises an organic solvent (os) and a compatible solute (cs) comprising the steps of:
   1) preparing the soaking solution by mixing os and cs
   2) transferring at least one crystal of the biological macromolecular crystal into the solution prepared in step 1) or transferring each solution prepared in step 1) into a compartment comprising at least one crystal of the biological macromolecular crystal
   3) controlling the crystal
   4) selecting the soaking solution which is suitable for soaking the crystal.
3. Method of selecting the composition of a non-aqueous soaking solution for soaking the crystal form of a biological macromolecule wherein the soaking solution comprises an organic solvent (os) and a compatible solute (cs); comprising the steps of:
   1) preparing a first series of 1n to xn individual soaking solutions by mixing os and cs, wherein:
      a) x is at least 2
      b) each soaking solutions of the first series comprises the same organic solvent (os) and the same compatible solute (cs
      c) Vos and Vcs are changed in each solution of the series, and
         in the solution 1n, Vₒₛ is the minimum Vₒₛ (Vₒₛmin) and the V_{cs} is the maximum V_{cs} (V_{cs}max), and
         in the solution xn, V_{cs} is the minimum V_{cs} (V_{cs}min) and the Vₒₛ is the maximum Vₒₛ (Vₒₛmax), and
         when x>2, in any additional solutions between 1n and xn, the Vₒₛ is varied between Vₒₛmin and Vₒₛmax and, inversely, Vcs is varied between V_{cs}min and V_{cs}max
   2) transferring at least one crystal of the biological macromolecular crystal into y compartments, each compartment comprising one of the solutions prepared in step 1 or transferring each solution prepared in step 1 into y compartments, each compartment comprising at least one crystal of the biological macromolecular crystal from its crystallization solution.
   3) controlling the crystal in each compartment
   4) selecting the soaking solution(s) which is/are suitable for soaking the crystal.
4. Method of selecting the composition of a non-aqueous soaking solution of embodiment 3 further comprising the step of:
   1) preparing m series of 1n to yn individual soaking solutions by mixing os and cs wherein m is at least 1 and wherein in each of the m series
      a) y is at least 2
      b) os is the same as in the first series, and
      c) the cs is changed and is different from the cs of the first series, and
      wherein in each individual solutions of each of the m series
      d) Vos and Vcs are changed in each solution, and
         in the solution 1n, Vₒₛ is the minimum Vₒₛ (Vₒₛmin) and the Vₒₛ is the maximum V_{cs} (V_{cs}max), and
         in the solution yn, V_{cs} is the minimum V_{cs} (V_{cs}min) and the Vₒₛ is the maximum Vₒₛ (Vₒₛmax), and
         when y>2, in any additional solutions between 1n and yn, the Vₒₛ is varied between Vₒₛmin and Vₒₛmax and, inversely, Vₒₛ is varied between Vosmin and Vₒₛmax
   2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.
5. Method of selecting the composition of a non-aqueous soaking solution of embodiment 3 or 4 further comprising the step of:
   1) preparing m series of 1n to yn individual soaking solutions by mixing os and cs wherein m is at least 1 and wherein in each of the m series
      a) y is at least 2
      b) cs is the same as in the first series, and
      c) the os is changed and is different from the os of the first series, and wherein in each individual solutions of each of the m series
      d) Vos and Vcs are different in each solution, and
         in the solution 1n, Vₒₛ is the minimum Vₒₛ (Vosmin) and the Vₒₛ is the maximum V_{cs} (V_{cs}max), and
         in the solution yn, Vₒₛ is the minimum Vcs (Vcsmin) and the Vₒₛ is the maximum Vₒₛ (Vₒₛmax), and
         when y>2, in any additional solutions between 1n and yn, the Vₒₛ varied between Vₒₛmin and Vₒₛmax and, inversely, V_{cs} is varied between V_{cs}min and Vcsmax
   2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.
6. Method of selecting the composition of a non-aqueous soaking solution of any of embodiment 3 to 5 wherein Vosmin and Vosmin are more than 0%.
7. Method of selecting the composition of a non-aqueous soaking solution of any of the embodiments 1-6 wherein the step of controlling the crystal is performed by X-Ray-diffraction neutron diffraction, absorption spectroscopy or reflectance spectroscopy in ultraviolet and visible spectral regions, infrared spectroscopy and/or visual analysis.
8. Method of selecting the composition of a non-aqueous soaking solution of any of the embodiments 1-7 wherein the os contains small molecules including molecule fragments and molecular probes.
9. Method of small molecule screening for a biological macromolecular crystal wherein the screening is performed in a soaking solution selected according to the method of embodiment 8.
10. Soaking solution obtainable by the method of selecting the composition of a non-aqueous soaking solution as described in any of the embodiments 1-8
11. Soaking solution as described in embodiment 10 wherein Vos is at least 5%.
12. Use of the soaking solution as described in any of embodiment 10 or 11 for small molecule screening for a biological macromolecular crystal.
13. Array arranged to perform the method as described in any of embodiments 1-9.
14. Array containing the solutions as described in any of embodiment 10 or 11.
15. Array containing non-aqueous solutions comprising an organic solvent (os) and a compatible solute (cs), the solution being prepared according to step 1) of any of embodiments 2 to 5.

The following embodiments are also part of the present invention:
1. Method of selecting the composition of a non-aqueous soaking solution suitable for soaking the crystal form of a biological macromolecule wherein the solution comprises an organic solvent (os) and a compatible solute (cs) comprising the steps of:
   1) preparing the soaking solution by mixing os and cs
   2) transferring at least one crystal of the biological macromolecular crystal into the solution prepared in step 1) or transferring each solution prepared in step 1) into a compartment comprising at least one crystal of the biological macromolecular crystal
   3) controlling the crystal
   4) selecting the soaking solution which is suitable for soaking the crystal; and
   wherein the compatible solute is selected from the group comprising polyols, amino acids, methylamines, and mixtures thereof, and
   wherein the organic solvent is selected from the group comprising liquid carbohydrates, protic or aprotic, of low reactivity, that can serve to solve small molecules and, mixtures thereof.
2. Method of selecting the composition of a soaking solution of embodiment 1, wherein the compatible solute is selected from the group comprising polyols, amino acids, methylamines or mixtures thereof, wherein the polyols are selected from a group comprising poly(oxyethylene), monosaccharide, disaccharide, trisaccharide, sugar alcohols, cyclitols and derivatives of former molecules, and wherein the amino acids are selected from a group comprising 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), ß-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[(1R)-1-carboxyethyl]amino]-5-(diaminomethylideneamino)pentanoic acid (Octopin) and wherein the methylamines are selected from a group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine).
3. Method of selecting the composition of a soaking solution of embodiment 1 or 2, wherein the compatible solute is selected from the group comprising polyols, amino acids, methylamines or mixtures thereof, wherein the polyols are selected from a group comprising poly(oxyethylene), polyhydric alcohols, monosaccharide, disaccharide, trisaccharide, sugar alcohols, cyclitols and derivatives of former molecules wherein the group of poly(oxyethylene) is selected from a group comprising monodisperse or polydisperse poly(oxyethylene) of molecular weights ranging between 20-2000000 g/mol and includes but is not limited to for example polyethylene glycol 200, polyethylene glycol 400, polyethylene glycol 600, and wherein the group of polyhydric alcohols is selected from a group comprising propane-1,2,3-triol (glycerol), ethane-1,2-diol (ethylene glycol), 2-Methylpentane-2,4-diol (2-Methylpentane-2,4-diol), (3R,4S,5S,6R)-2-(2,3-dihydroxypropoxy)-6-(hydroxymethyl)oxane-3,4,5-triol (1-Glucosylglycerol) and wherein the group of monosaccharide is selected from the group comprising (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside) and wherein the group of disaccharides is selected from the group comprising β-D-Fructofuranosyl α-D-glucopyranoside (sucrose), (2R,3S,4S,5R,6R)-2-(Hydroxymethyl)-6-[(2R,3R,4S,5S,6R)-3,4,5-trihydroxy-6-(hydroxymethyl)oxan-2-yl]oxyoxane-3,4,5-triol (trehalose) and wherein the group of trisaccharides is selected from the group comprising (2R,3R,4S,5S,6R)-2-[(2S,3S,4S,5R)-3,4-dihydroxy-2,5-bis(hydroxymethyl)oxolan-2-yl]oxy-6-[[(2S,3R,4S,5R,6R)-3,4,5-trihydroxy-6-(hydroxymethyl)oxan-2-yl]oxymethyl]oxane-3,4,5-triol (raffinose) and wherein the group of sugar alcohols comprises (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol) and (2R,3S,4R,5S)-hexane-1,2,3,4,5,6-hexol (galactitol)) and wherein the group of cyclitols comprises (1S,2S,4S,5R)-6-methoxycyclohexane-1,2,3,4,5-pentol (pinitol) and (2S,3R,4S,5R,6R)-2-[(1,3-dihydroxypropan-2-yl)oxy]-6-(hydroxymethyl)oxane-3,4,5-triol (fluoridoside) and wherein the group of derivatives of former molecules comprises mannose derivatives as fiorin ((2R)—O2-(β-D-mannopyranosyl)glyceric acid) and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose.
4. Method of selecting the composition of a soaking solution according to any of embodiments 1 to 3, wherein the organic solvent is a straight or branched chain monohydric aliphatic alcohol, dihydric aliphatic alcohol, monoalkyl ether of an aliphatic dihydric alcohol, ether of an aliphatic monohydric alcohol, cyclic non-aromatic ethers, (cyclic) enol-ethers, dialkyl ketones, benzyl alcohols, phenyl ethyl alcohols, (alkylated) sulfoxides and according phosphoderivatives, esters derived from acetic acid, (alkylated) formamides or mixtures thereof.
5. Method of selecting the composition of a soaking solution according to any of embodiments 1 to 4, wherein said soaking solution comprises an organic solvent (os) and a compatible solute (cs),
   wherein said compatible solute (cs) is a polyol or a mixture thereof, and said organic solvent (os) is a liquid carbohydrate protic or aprotic, of low reactivity, or a mixture thereof, that can serve to solve small molecules,
   more preferably, said compatible solute (cs) is a poly(oxyethylene) and said organic solvent (os) is selected from the group comprising a (alkylated) sulfoxide, cyclic non-aromatic ether, straight or branched chain monohydric aliphatic alcohol, alkylated formamide or a mixture thereof,
   most preferably, the compatible solute cs is selected from the group comprising the poly(oxyethylene)s polyethylene glycol 200, polyethylene glycol 400, polyethylene glycol 600, or a mixture thereof and said organic solvent (os) is selected from the group comprising dimethyl sulfoxide, 1,4-dioxane, dimethylformamide, methanol, ethanol, or a mixture thereof.
6. Method of selecting the composition of a soaking solution according to any of embodiments 1 to 4, wherein said soaking solution comprises an organic solvent (os) and a compatible solute (cs),
   wherein said compatible solute (cs) is a polyol or a mixture thereof, and said organic solvent is a is a liquid carbohydrate protic or aprotic, of low reactivity, or a mixture thereof, that can serve to solve small molecules,
   more preferably, said compatible solute (cs) is a polyhydric alcohol and said organic solvent (os) is selected from the group comprising a (alkylated) sulfoxide, cyclic non-aromatic ether, straight or branched chain monohydric aliphatic alcohol, alkylated formamide or a mixture thereof,
   most preferably, the compatible solute (cs) is selected from the group comprising the polyhydric alcohols propane-1,2,3-triol (glycerol), ethane-1,2-diol (ethylene glycol), 2-Methylpentane-2,4-diol (MPD), (3R,4S,5S,6R)-2-(2,3-dihydroxypropoxy)-6-(hydroxymethyl)oxane-3,4,5-triol (1-Glucosylglycerol) or a mixtures thereof, and said organic solvent (os) is selected from the group comprising dimethyl sulfoxide, 1,4-dioxane, dimethylformamide, methanol, ethanol, or a mixture thereof.
7. Method of selecting the composition of a soaking solution according to any of embodiments 1 to 4, wherein said soaking solution comprises an organic solvent (os) and a compatible solute (cs),
   wherein said compatible solute (cs) is a polyol a or a mixture thereof, and said organic solvent (os) is a liquid carbohydrate protic or aprotic, of low reactivity, or a mixture thereof, that can serve to solve small molecules,
   more preferably said compatible solute (cs) is a mono-, di-, tri, oligo- or polysaccharide or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
   most preferably, the compatible solute (cs) is selected from the group comprising the saccharides (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) or β-D-Fructofuranosyl α-D-glucopyranoside (sucrose) or a mixtures thereof, and said organic solvent (os) is selected from the group comprising dimethyl sulfoxide, 1,4-dioxane, dimethylformamide, methanol, ethanol, or a mixture thereof.
8. Method of selecting the composition of a soaking solution according to any of embodiments 1 to 4, wherein said soaking solution comprises an organic solvent (os) and a compatible solute (cs),
   wherein said compatible solute (cs) is a L- or D amino acid, or a mixture thereof, and the organic solvent (os) is liquid carbohydrate protic or aprotic, of low reactivity, or a mixture thereof, that can serve to solve small molecules,
   more preferably said compatible solute (cs) is a L-amino acid, or a mixture thereof, and said organic solvent is a (alkylated) sulfoxide, cyclic non-aromatic ether, straight or branched chain monohydric aliphatic alcohol, alkylated formamide, or a mixture thereof,
   most preferably, said compatible solute (cs) is selected from the group comprising the L-amino acids (2S)-pyrrolidine-2-carboxylic acid (proline), 2-aminoacetic acid (glycine), (6S)-2-methyl-1,4,5,6-tetrahydropyrimidine-6-carboxylic acid (ectoine) or (5S,6S)-5-hydroxy-2-methyl-1,4,5,6-tetrahydropyrimidine-6-carboxylic acid (hydroxyectoine), or a mixture thereof, and said organic solvent (os) is selected from the group comprising dimethyl sulfoxide, 1,4-dioxane, dimethylformamide, methanol, ethanol, or a mixture thereof.
9. Method of selecting the composition of a soaking solution according to any of embodiments 1 to 4, wherein said soaking solution comprises an organic solvent (os) and a compatible solute (cs),
   wherein said compatible solute (cs) is a methylamine or a mixture thereof and said organic solvent (os) is liquid carbohydrate protic or aprotic, of low reactivity, or a mixture thereof that can serve to solve small molecules,
   more preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is a (alkylated) sulfoxide, cyclic non-aromatic ether, straight or branched chain monohydric aliphatic alcohol, alkylated formamide, or a mixture thereof,
   most preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is selected from the group comprising dimethyl sulfoxide, 1,4-dioxane, dimethylformamide, methanol, ethanol, or a mixture thereof.
10. Method of selecting the composition of a soaking solution according to any of embodiments 1 to 4, wherein said soaking solution comprises an organic solvent (os) and a compatible solute (cs), and
   wherein said compatible solute (cs) is polyethylene glycol 400 and said organic solvent (os) is dimethyl sulfoxide.
11. Method of selecting the composition of a soaking solution according to any of embodiments 1 to 4 and 6, wherein said soaking solution comprises an organic solvent (os) and a compatible solute (cs),
   wherein said compatible solute (cs) is ethane-1,2-diol (ethylene glycol) and said organic solvent (os) is dimethyl sulfoxide.
12. Method of selecting the composition of a soaking solution according to any of embodiments 1 to 4 and 6, wherein said soaking solution comprises an organic solvent (os) and a compatible solute (cs),
   wherein said compatible solute (cs) is propane-1,2,3-triol (glycerol) and said organic solvent (os) is dimethyl sulfoxide.
13. Method of selecting the composition of a soaking solution according to any of embodiments 1 to 4 and 6, wherein said soaking solution comprises an organic solvent (os) and a compatible solute (cs),
   wherein said compatible solute (cs) is 2-methylpentane-2,4-diol (MPD) and said organic solvent (os) is dimethyl sulfoxide.
14. Method of selecting the composition of a non-aqueous soaking solution for soaking the crystal form of a biological macromolecule wherein the soaking solution comprises an organic solvent (os) and a compatible solute (cs); comprising the steps of:
   1) preparing a first series of 1n to xn individual soaking solutions by mixing os and cs, wherein:
   a) x is at least 2
   b) each soaking solutions of the first series comprises the same organic solvent (os) and the same compatible solute (cs)
   c) Vos and Vcs are changed in each solution of the series, and
      in the solution 1n, Vₒₛ is the minimum Vₒₛ (Vₒₛmin) and the V_{cs} is the maximum V_{cs} (V_{cs}max), and
      in the solution xn, V_{cs} is the minimum V_{cs} (V_{cs}min) and the Vₒₛ is the maximum Vₒₛ (Vₒₛmax), and
      when x>2, in any additional solutions between 1n and xn, the Vₒₛ is varied between Vosmin and Vₒₛmax and, inversely, Vcs is varied between Vosmin and Vcsmax;
   or, alternatively, Vₒₛ and N_{cs} are changed in each solution of the series, and
      in the solution 1n, Vₒₛ is the minimum Vₒₛ (Vₒₛmin) and the Nos is the maximum N_{cs} (N_{cs}max), and
      in the solution xn, N_{cs} is the minimum N_{cs} (N_{cs}min) and the Vₒₛ is the maximum Vₒₛ (Vₒₛmax), and
      when x>2, in any additional solutions between 1n and xn, the Vₒₛ is varied between Vₒₛmin and Vₒₛmax and, inversely, N_{cs} is varied between Nosmin and N_{cs}max,
   2) transferring at least one crystal of the biological macromolecular crystal into y compartments, each compartment comprising one of the solutions prepared in step 1 or transferring each solution prepared in step 1 into y compartments, each compartment comprising at least one crystal of the biological macromolecular crystal from its crystallization solution.
   3) controlling the crystal in each compartment
   4) selecting the soaking solution(s) which is/are suitable for soaking the crystal.
15. Method of selecting the composition of a non-aqueous soaking solution of embodiment 14 further comprising the step of:
   1) preparing m series of 1n to yn individual soaking solutions by mixing os and cs wherein m is at least 1 and wherein in each of the m series
      a) y is at least 2
      b) os is the same as in the first series, and
      c) the cs is changed and is different from the cs of the first series, and
      wherein in each individual solutions of each of the m series
      d) Vₒₛ and Vcs are changed in each solution, and
         in the solution 1n, Vₒₛ is the minimum Vₒₛ (Vₒₛmin) and the V_{cs} is the maximum V_{cs} (V_{cs}max), and
         in the solution yn, Vₒₛ is the minimum V_{cs} (V_{cs}min) and the Vₒₛ is the maximum Vₒₛ (Vₒₛmax), and
         when y>2, in any additional solutions between 1n and yn, the Vₒₛ is varied between Vosmin and Vₒₛmax and, inversely, Vₒₛ is varied between Vosmin and Vcsmax,
         or, alternatively, Vₒₛ and N_{cs} are changed in each solution, and
         in the solution 1n, Vₒₛ is the minimum Vₒₛ (Vₒₛmin) and the Nos is the maximum N_{cs} (N_{cs}max), and
         in the solution yn, N_{cs} is the minimum N_{cs} (N_{cs}min) and the Vₒₛ is the maximum Vₒₛ (Vₒₛmax), and
         when y>2, in any additional solutions between 1n and yn, the Vₒₛ is varied between Vₒₛmin and Vₒₛmax and, inversely, N_{cs} is varied between Nₒₛmin and Vₒₛmax,
   2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.
16. Method of selecting the composition of a non-aqueous soaking solution of embodiment 14 or 15 further comprising the step of:
   1) preparing m series of 1n to yn individual soaking solutions by mixing os and cs wherein m is at least 1 and wherein in each of the m series
      a) y is at least 2
      b) cs is the same as in the first series, and
      c) the os is changed and is different from the os of the first series, and
      wherein in each individual solutions of each of the m series
      d) Vₒₛ and Vcs are different in each solution, and
         in the solution 1n, Vₒₛ is the minimum Vₒₛ (Vₒₛmin) and the Vₒₛ is the maximum V_{cs} (V_{cs}max), and
         in the solution yn, Vₒₛ is the minimum V_{cs} (V_{cs}min) and the Vₒₛ is the maximum Vₒₛ (Vₒₛmax), and
         when y>2, in any additional solutions between 1n and yn, the Vₒₛ varied between Vₒₛmin and Vₒₛmax and, inversely, Vₒₛ is varied between Vosmin and V_{cs}max,
         or, alternatively, Vos and Ncs are different in each solution, and
         in the solution 1n, Vₒₛ is the minimum Vₒₛ (Vₒₛmin) and the N_{cs} is the maximum N_{cs} (N_{cs}max), and
         in the solution yn, N_{cs} is the minimum N_{cs} (N_{cs}min) and the Vₒₛ is the maximum Vₒₛ (Vₒₛmax), and
         when y>2, in any additional solutions between in and yn, the Vₒₛ varied between Vₒₛmin and Vₒₛmax and, inversely, N_{cs} is varied between N_{cs}min and N_{cs}max,
   2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.
17. Method of selecting the composition of a non-aqueous soaking solution of any of embodiment 14 to 16 wherein Vosmin and Vosmin are more than 0%.
18. Method of selecting the composition of a non-aqueous soaking solution of any of the embodiments 1 to 17 wherein the step of controlling the crystal is performed by X-Ray-diffraction neutron diffraction, absorption spectroscopy or reflectance spectroscopy in ultraviolet and visible spectral regions, infrared spectroscopy and/or visual analysis.
19. Method of selecting the composition of a non-aqueous soaking solution of any of the embodiments 1 to 18 wherein the os contains small molecules including molecule fragments and molecular probes to be analyzed, which diffuse into the biological macromolecular crystal.
20. Method of small molecule screening for a biological macromolecular crystal wherein the screening is performed in a soaking solution selected according to the method of embodiment 19.
21. Soaking solution obtainable by the method of selecting the composition of a non-aqueous soaking solution as described in any of the embodiments 1 to 18.
22 Soaking solution as described in embodiment 20 wherein Vos is at least 0.5%.
23. Use of the soaking solution as described in any of embodiment 20 or 21 for small molecule screening for a biological macromolecular crystal.
24. Array arranged to perform the method as described in any of embodiments 1 to 17, wherein said array comprises a first dimension of at least two individual non-aqueous soaking solutions (1n to xn) and a second dimension of at least two individual non-aqueous soaking solutions (1m to ym) wherein each of said soaking solutions is located in a separated compartment of said array, and wherein each of said soaking solution comprises an organic solvent (os) and a compatible solute (cs).
25. Array according to embodiment 24, wherein the array comprises of 1x to xn series in said first dimension of individual soaking solutions for the biological macromolecule wherein the compatible solute of each of the series of individual non-aqueous soaking solution in the first dimension differs from the one of the series of individual soaking solutions of the second dimension and the organic solvent of each of the series of individual non-aqueous soaking solution in the first dimension differs from the one of the series of individual soaking solutions of the second dimension.
26. Use of an Array according any of embodiments 24 to 25 for obtaining the solutions as described in embodiment 19 or 20.

### FIGURE DESCRIPTION

FIG. 1: 1A is a picture of a screening plate used for soaking selection and crystal preparation/production; FIG. 1B shows the details of one of the well of a 24 well plate and on FIG. 1C a schematic representation of the well during a soaking experiment or crystal preparation/production is depicted.

FIG. 2 shows a preparation scheme according to the method of the invention for selection of soaking solutions comprising CS: compatible solute and OS-organic solvent. The lines (compatible solute A-D) represent the first dimension and the columns (1-6) the second dimension

FIG. 3 shows an example of a scheme according to the method of the invention for selection of soaking solutions comprising for fragment screening comprising PEG400-polyethylene glycol 400; MPD-2-methylpentane-2,4-diol; EG-ethane-1,2-diol (ethylene glycol); Gly-propane-1,2,3-triol (glycerol); OS-organic solvent=DMSO.

FIG. 4 shows an example of the visual difference between a suitable crystal (A) and crystals suffering from a severe quality loss (B) due to detrimental soaking conditions/solution

FIG. 5 shows a template for the visual inspection of crystals under i.e. a microscope.

FIG. 6 shows the criteria used for visual inspection and to control and select the solutions having minimal impact on the crystal

FIG. 7 shows an image of a diffraction pattern after an X-Ray experiment.

FIG. 8 shows a template for the X-Ray inspection of crystals.

FIG. 9 shows the criteria used for X-Ray inspection and to control and select the solutions having minimal impact on the crystal.

FIG. 10 shows a template scheme for fragment screening.

FIG. 11 shows an example of potential workflow representing the different step of the processes of the invention performed in a soaking selection experiment with subsequent fragment screening.

FIG. 12 shows IspD that was not subjected to a waterfree soaking experiment in a surface representation. The black spheres on the surface represent immobilized water molecules, so called structure water.

FIG. 13 shows IspD that was subjected to a waterfree soaking experiment in a surface representation. The black spheres on the surface represent immobilized water molecules, so called structure water.

FIG. 14 shows details of IspD's active site that was not subjected to a waterfree soaking.

FIG. 15 shows details of IspD's active site that was subjected to a waterfree soaking.

FIG. 16 shows IspD that was not subjected to a waterfree soaking experiment in a surface representation. Left—The dark grey colour indicates the distribution of negative charges; Right—The dark grey colour indicates the distribution of positive charges.

FIG. 17 shows IspD that was subjected to a waterfree soaking experiment in a surface representation. Left—The dark grey colour indicates the distribution of negative charges; Right—The dark grey colour indicates the distribution of positive charges.

FIG. 18 shows the excerpt of a reconstructed electron density.

FIG. 19 shows the same as FIG. 18 but completed with the protein's amino acid sequence and structurally fixed water molecules (dots).

FIG. 20 shows an example of electron density

FIG. 21 shows an example of electron density together with according amino acids and water molecules (spheres).

FIG. 22 shows an example of unassigned electron density

FIG. 23 shows an example of an electron density which could be assigned to a small molecule fragment as it was used in a small molecule fragment screening.

### EXAMPLES

### Example 1: Water Structure Analysis on the Protein IspD

### a) Crystallization of IspD

First a IspD solution of a concentration of 39 mg/ml in a buffer made of 50 mM Tris, pH 7.3, 100 mM NaCl and 5 mM DTT was prepared as well as a reservoir solution of 20% (w/v) polyethylene glycol 3350 and 180 mM tri-ammonium citrate (triazanium;2-hydroxypropane-1,2,3-tricarboxylate). Crystallization was performed by the vapor-diffusion method at 18° C. using 4 μl of a mixture of these two solutions in a ratio of 1:1 (v/v) against 1000 μl of the reservoir buffer mentioned above in the reservoir.

### b) Determination of Waterfree-Soaking Environment for IspD by the Application of Waterfree.

IspD crystals were harvested via a cryo-loop from the crystallization plate and transferred to the wells of a beforehand prepared 24-well experimental plate. On this experimental plate a soaking solution was prepared that had been determined beforehand by following an a method of selection claimed in a separate patent application with the same title, filed on the same date and in the name of the same applicant as the present patent application. In this alternative or complementary, the soaking solution is selected using a similar method. However one of the key differences is that the soaking solution used in the other method is aqueous i.e. contains water. The crystals were conditioned in this aqueous soaking solution for 1-48 hours. Afterwards crystals were fished using a cryo-loop and transferred to each well of another 24-well experimental plate that was prepared according to the rules of the non aqueous soaking solution-protocol (FIG. 3) as follows:

Preparation of a first series of 6 individual soaking solutions by mixing os and cs, wherein each soaking solutions of the first series comprises the same organic solvent (os) (DMSO) and the same compatible solute (cs) (for instance the first line with PEG400) but the volume/proportion of Vos and Vos are changed in each solution of the series, and one of the solution contains Vₒₛmin is 20% (1 □l) and the V_{cs}max is 80% (4 □l) (see column 1), and another solution contains of has V_{cs}min is 30% (1.5 □l) and Vₒₛmax is 70% (3.5 □l) (see column 6) and in the other solutions (see columns 2-5) the Vₒₛ is varied between Vosmin and Vₒₛmax and, inversely, Vcs is varied between V_{cs}min and V_{cs}max. In this example, V_{cs} is incrementally varied between V_{cs}min and V_{cs}max and, inversely, Vₒₛ is incrementally varied between Vₒₛmin and Vₒₛmax in any additional solutions (see column 2-5 of line PEG400) and in any additional solutions, the increment of Vₒₛ between V_{cs}min and V_{cs}max and of Vₒₛ between Vₒₛmin and Vₒₛmax remains the same (10%) between each solution of the first series.

The solutions were distributed in the wells of a line (line PEG400 on FIG. 3) of an array thus constituting a first dimension. The solutions were preferably distributed following an order from the lowest Vos to the highest Vos but the order of the distribution in the array can be random.

Subsequently or simultaneously, the following step were performed as follows:

Preparation of 3 (m) additional series of 6 individual non-aqueous soaking solutions by mixing os and cs. In this example, the number of individual soaking solutions 6 is conserved for each series and is the same as the number of individual soaking solutions of the first series. In each additional series, os is the same as in the first series (DMSO), but in each series the cs is changed (MPD, EG, Glycerol) and is different from the cs of the first series (PEG400). Within a given series, Vos and Vcs are changed in each solution as follows: each soaking solutions of each of the additional series comprises the same organic solvent (os) (DMSO) and the same compatible solute (cs) (for instance the second line with MPD) but the volume/proportion of Vₒₛ and Vcs are changed in each solution of the series, and one of the solution is composed of Vosmin is 20% (1□l) and the Vcsmax is 80% (4 □l) (see column 1), and another solution is composed of has V_{cs}min is 30% (1.5 □l) and Vₒₛmax is 70% (3.5 □l) (see column 6) and in the other solutions (see columns 2-5) the Vos is varied between Vosmin and Vosmax and, inversely, Vcs is varied between Vcsmin and Vcsmax. In this example, Vcs is incrementally varied between Vcsmin and Vcsmax and, inversely, Vos is incrementally varied between Vosmin and Vcsmax in any additional solutions (see column 2-5 of line MPD, EG and Glycerol) and in any additional solutions, the increment of Vcs between Vosmin and Vcsmax and of Vos between Vosmin and Vosmax remains the same (10%) between each solution of the first series.

The solutions were distributed in the wells of additional line (line MPD, EG and Glycerol on FIG. 3) of an array thus constituting a second dimension. The solutions were preferably distributed following an order from the lowest Vos to the highest Vos but the order of the distribution in the array can be random.

Then at least one IspD crystal prepared in a) above was placed in each compartments of the array of FIG. 3. The crystal was placed under microscope for control by visual analysis.

After 5 days the crystals, the crystals in all but one well were dissolved. Thus, one non-aqueous environment was selected. The selected non-aqueous solution is composed of 60% PEG400 and 40% DMSO (column 3 line PEG400 of FIG. 3). A crystal conditioned in this water-free environment was fished, vitrified and subjected to measurements at a synchrotron as well as an IspD crystal from the same treatment but with the above referred aqueous soaking solution for comparison.

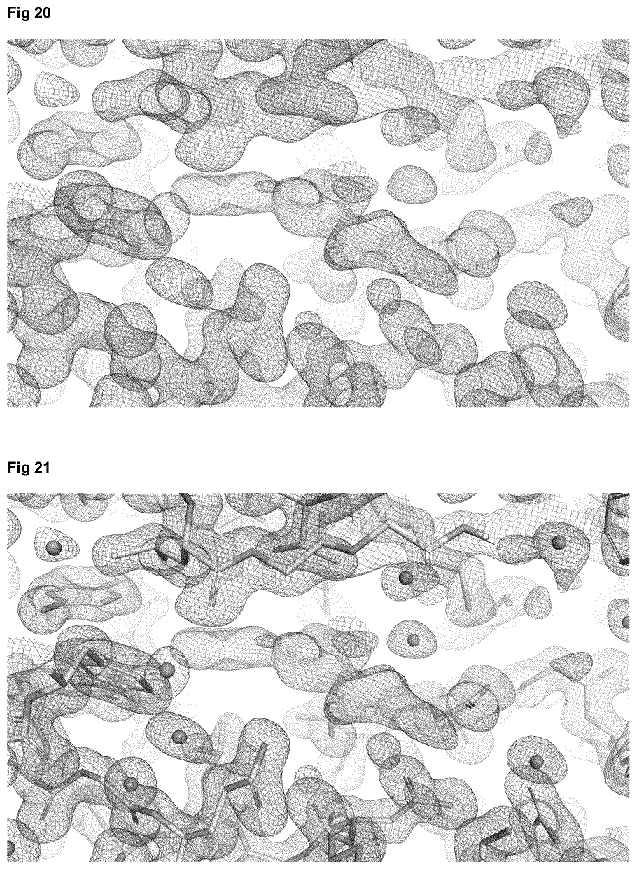

The measurements yielded two complete data sets. The crystal from the water-free environment diffracted at a resolution of 1.28 Å while the reference from the aqueous environment diffracted at a resolution of 1.32 Å. FIG. 20 shows the pure electron density as reconstructed from the raw data of the protein from the waterfree environment and FIG. 21 the according electron density with the according amino acids assigned to the empty electron densities. Data evaluation resulted in the identification of 49 observable water molecules for the crystal from the water-free environment and 182 observable water molecules for the crystal from the aqueous environment. This is a striking difference and shows that only 30% of waters are highly immobilized while 70% are eliminated in a waterfree environment (Table 1).

TABLE 1
comparison of both proteins

| | | | | | HYDROPHOBIC | HYDROPHOBIC | | |
|---|---|---|---|---|---|---|---|---|
| | Number of | | Positive | Negative | ACCESSIBLE | ACCESSIBLE | | Dipole |
| | structure | Net | patches | patches | SURFACE AREA | SURFACE AREA | Volume | moment |
| | waters | charge | [Å2] | [Å2] | [Å2] | [Å2] | [Å3] | [D]r |
| IspDA | 182 | −8.03 | 430 | 640 | 5787.55 | 4280.39 | 20671.25 | 369.10 |
| IspDw | 49 | −16.76 | 360 | 710 | 6217.07 | 3969.94 | 22030.25 | 652.73 |

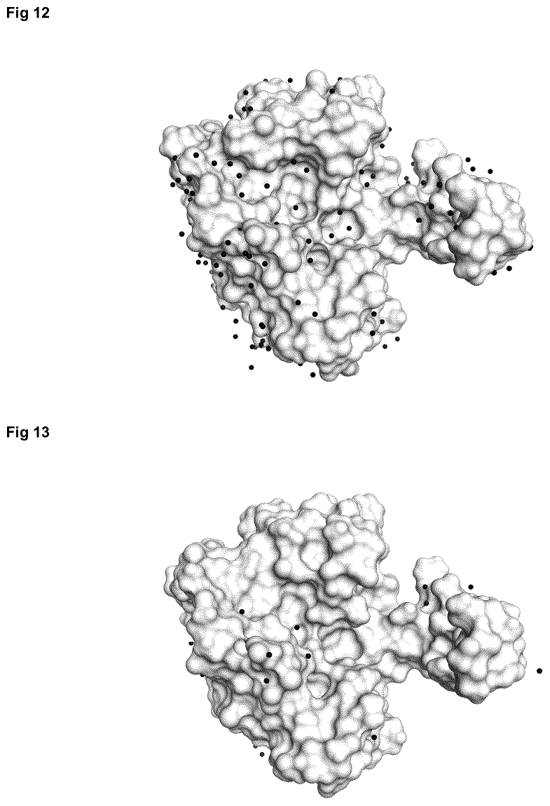

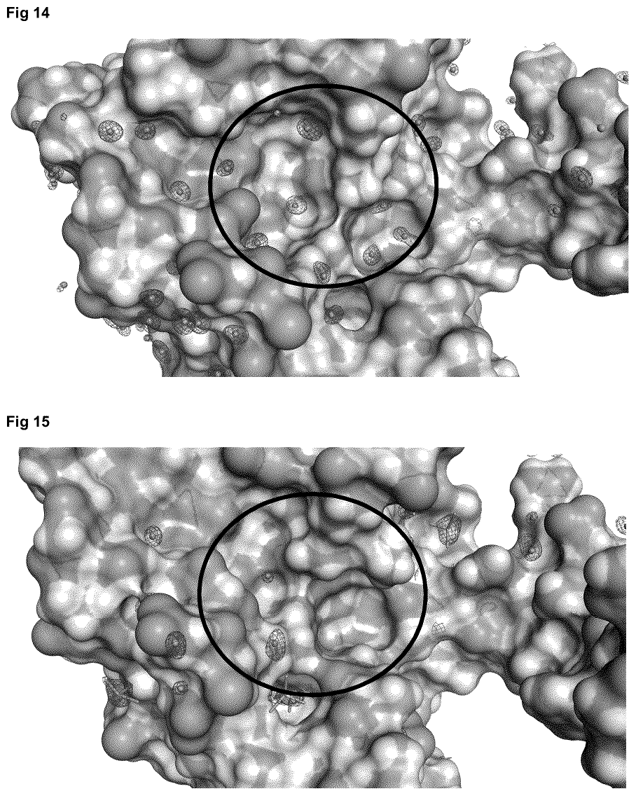

FIG. 12 and FIG. 13 show the location of respective water molecules (black dots) on the surface of both samples. FIG. 14 and FIG. 15 show in detail the difference of immobilized waters (dots) in the area of the binding pocket.

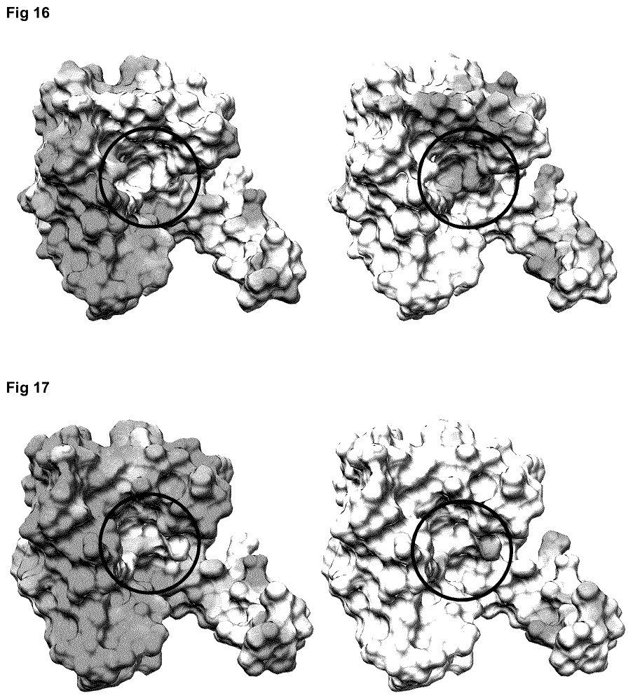

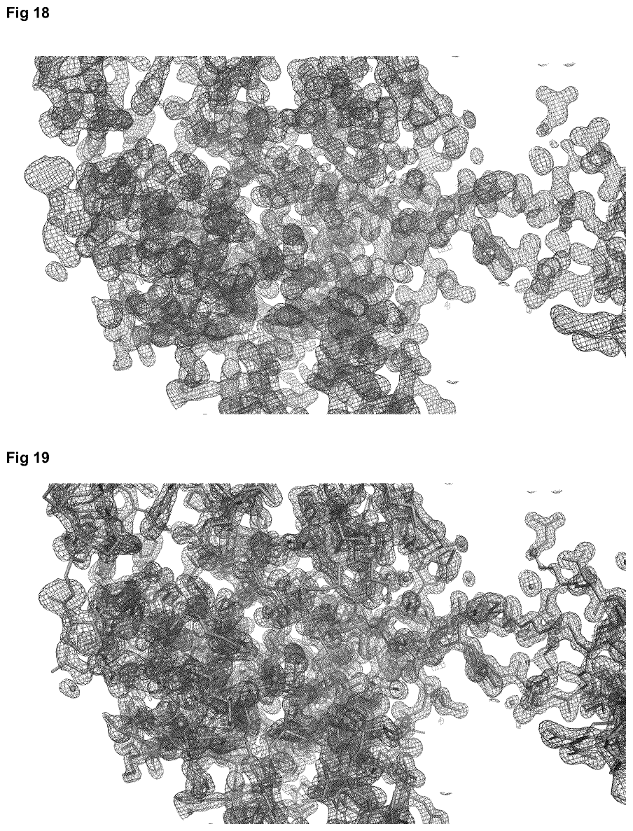

Interestingly, the two structures still show a high degree of alignment if superimposed. Nevertheless, the volume of the two proteins differs around 6% (Table. 1). A comparison of the B-factors of the amino acids on the surface of the protein shows a lower degree of flexibility for the waterfree structure (FIG. 16 and FIG. 17). At the same time the hydrophobic accessible surface area increases around 7% in the waterfree sample while the hydrophilic accessible surface area of the waterfree sample is decreased by 7% (Tab. 1). This clearly shows that geometrical properties on the surface change when proteins are transferred to waterfree environments. Furthermore, the distribution of charges on the surface changes (colored pictures not shown). The protein treated by a waterfree soaking has an overall double negative net charge as the protein from the aqueous environment (Table 1).

This corresponds to a reduced positive surface area in the waterfree environment and an increased negative surface area (Table 1).

By observing a surface representation of the protein (coloured pictures not shown), a decrease in the positive charged surface area around the active site can be seen. Information about the charge distribution can be of relevance for library design. Knowledge about the locations of waters that can be replaced and that cannot be replaced can facilitate compound evolution.

The study is just one example of a very successful application of non-aqueous soaking solution for the purpose of an analysis of structure water. In the same way non aqueous soaking solution is applicable to identify water-free soaking conditions for screenings or soakings of hydrophobic small molecules insoluble in aqueous environments. Non aqueous soaking solution can even be utilized to dry crystals at ambient conditions for storage and transportation or to improve diffraction quality. That proves that the non-aqueous soaking solution-technology is a very successful means for a variety of applications.

### Example 2

Other applications of establishing a water-free environment for a macromolecular crystal are soaking of hydrophobic small molecules that are not solvable in aqueous environments or the controlled dehydration of macromolecular crystals in order to improve the crystal's diffraction quality. Macromolecular crystals from a water-free environment can be used for diffraction experiments in two ways: They can be obtained from the water-free environment and used for diffraction experiments at room temperature or at cryogenic temperature still embedded in a drop of liquid from the water-free environment or they can be dried under for example ambient conditions and used afterwards for diffraction experiments. It has been shown that such crystals tolerate even harsh conditions like sputtering with gold for subsequent analysis by scanning electron microscopy. Further applications are to transfer crystals into a sustainable storage and transportation form.

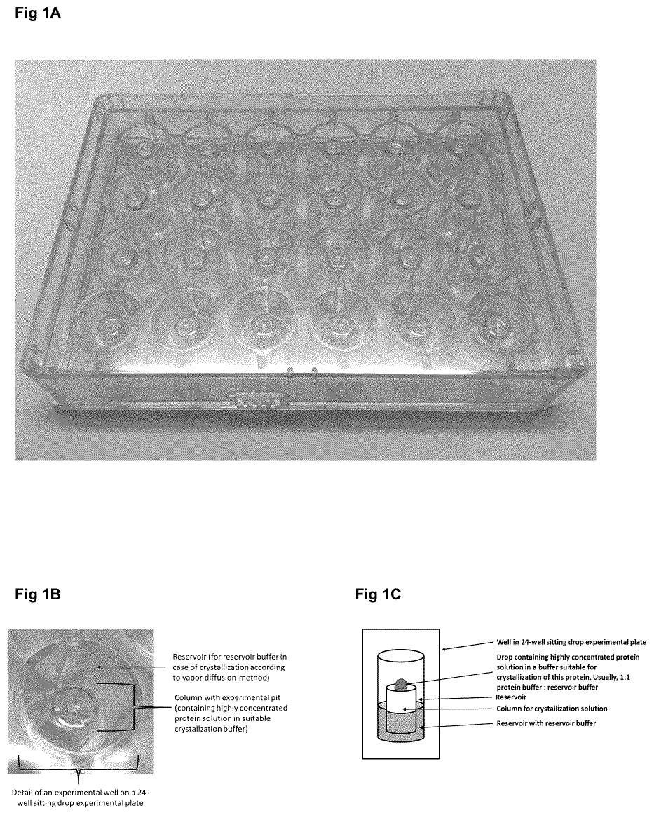

In order to perform a “non-aqueous soaking solution”-based small molecule fragment screening on macromolecular crystals, crystals of the protein of interest (POI) must be obtained (step a of workflow in FIG. 11). In the example, the crystallization is conducted according to the vapor diffusion approach in commercially available 24-well sitting drop crystallization plates (or any other plate format) (FIG. 1A). For this purpose, the reservoirs of each 24-well plate used are filled with a buffer according to a beforehand established crystallization protocol. Furthermore, a crystallization drop is prepared in the indentation on top of the column of each of the wells by adding some volume of a protein solution to some volume of the buffer which is also used in the reservoir. Usually, but not always the volumes are in a ratio of 1:1 (FIGS. 1B and 1C). The resulting protein concentration is usually, but not always, in the range of 2 to 30 mg/ml but can also reach concentrations of 250 mg/ml or more. Afterwards the wells are sealed using an adhesive film, so called sealing tape. All these steps are conducted in an according to the crystallization protocol correctly tempered laboratory, usually at 4° C., 10° C., 18° C., 20° C. The plates are stored on a shelf in this laboratory for the time needed for crystallization, usually a few days to a few weeks.

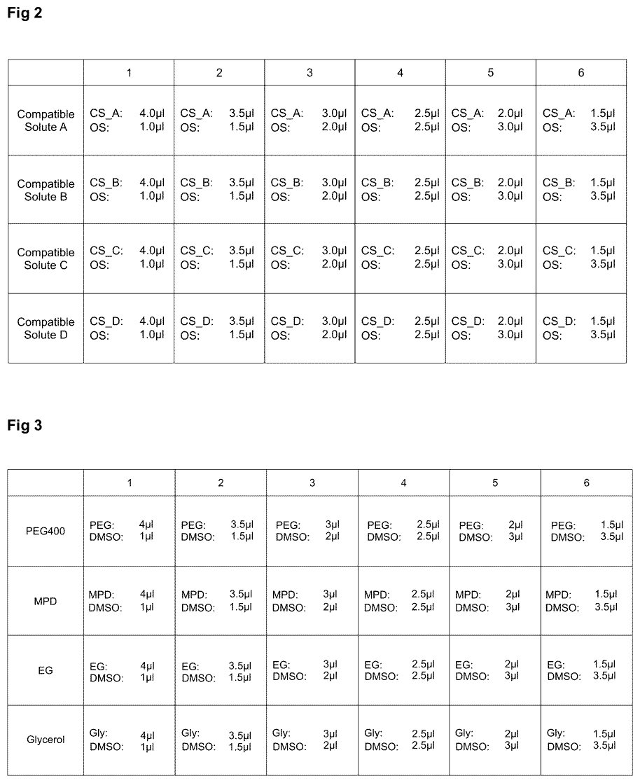

After the crystallization process is finished, a Non aqueous soaking solution-screening according to the present invention is conducted in order to establish a water-free environment for the macromolecular crystal. For this purpose, a 24-well crystallization plate (or any other plate format) is utilized, and the indentation of each well are provided with possible soaking solutions according to the present invention as is exemplified in FIG. 2 (step b of workflow in FIG. 11). Subsequently, the film used for sealing the 24-well plates is removed from the plates used for crystallization of the POI. The meanwhile grown crystals are removed from their growth solutions using a special laboratory tool usually equipped with a nylon loop for crystal picking or any other appropriate tool for the so-called “crystal fishing” and transferred to the possible water-free environments in the indentations of the new 24-well plate that was prepared according to an instantiation of the on aqueous soaking solution-protocol (step c of workflow in FIG. 11). These plates are also sealed using sealing tape. In some cases, the macromolecular crystals require a conditioning step before the transfer to the Non aqueous soaking solution-screening: They are conditioned in one or more beforehand identified soaking-conditions in order to remove water stepwise. The purpose is to remove water stepwise by decreasing the water content and increasing the non-aqueous components of the soaking solution. This is performed in order to cushion osmotic shock occurring upon transfer of the macromolecular crystals into a water-free environment directly. Oftentimes the additives and organic solvents used in this respective aqueous soaking-condition or conditions are used to establish the water-free environment according to the protocol for non-aqueous soaking-screenings.

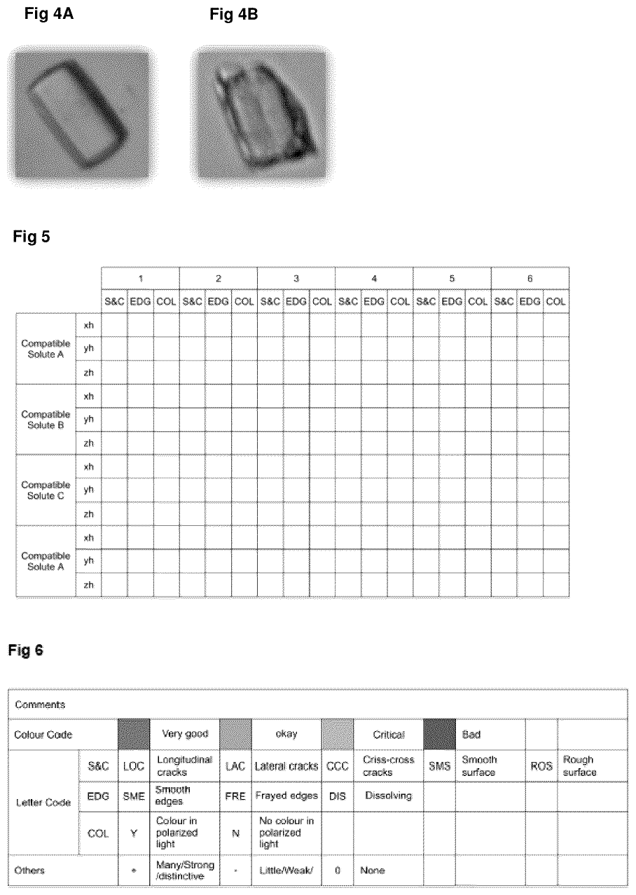

The purpose of the next steps is to evaluate the crystals quality after Non aqueous soaking solution-treatment, the maximal soaking time and a proper relation between soaking time and crystal quality for non-aqueous soaking solution applications. Within a certain time, usually the next 24 hours, pictures of each indentation are taken under a microscope at least 1 time, usually three times, sometimes more, under a microscope in order to document the crystals conditions for example immediately after the transfer, after 1 hour and after 24 hours. FIG. 4A exemplifies the visual appearance that a crystal usually shows when a soaking solution composition is suitable. FIG. 4B, in contrast, shows the appearance a protein crystal that was treated with an unsuitable soaking solution composition. In parallel, usually notes are taken in order to supplement the information in the pictures. A template for such notes is exemplified in FIG. 5. Notes refer to different types of cracks crystals can show (longitudinal, lateral, crisscross), to the appearance of the crystals surface (e.g. smooth, rough) or edges (e.g. sharp, roundish) and their behavior in polarized light (strong color, some color, no color). In the most cases not all of the crystals survive in all the conditions. Some crystals dissolve over time or are damaged while other crystals are in good condition. Therefore, a x-ray evaluation of the crystal's quality is obligatory (step d of workflow in FIG. 11).

In order to do this the macromolecular crystals can be handled in three different ways: After removing of the sealing tape from the plate remaining crystals can either be transferred from the plate into liquid nitrogen or another vitrification medium, or they can be directly subjected to a x-ray study at room temperature (without vitrification but embedded in a liquid drop), or they can be dried for example under ambient conditions. The crystals vitrification takes place in the nylon loops mentioned before or any other equivalent transfer-equipment like litho-loops or capillaries. The vitrified crystals in the nylon loops or other transfer-equipment are stored in so called “cryo vials” under liquid nitrogen or another vitrification medium until they are subjected to an x-ray examination. Crystals can also be stored in any other appropriate storage containers as, for example, Uni-Pucks. For the x-ray examination the crystals in the nylon loops or other transfer-equipment are removed from the cryo vials or other storage containers and mounted on an x-ray machine in a so called cryo-stream that maintains the cryogenic temperature around the protein crystal. In case of x-ray studies at room temperature, no cryo-stream is required. In case of crystal-drying the crystals are mounted on the x-ray machine in any suitable way for example by fixing the dried crystals in the known nylon loops using spider silk or any other suitable means.

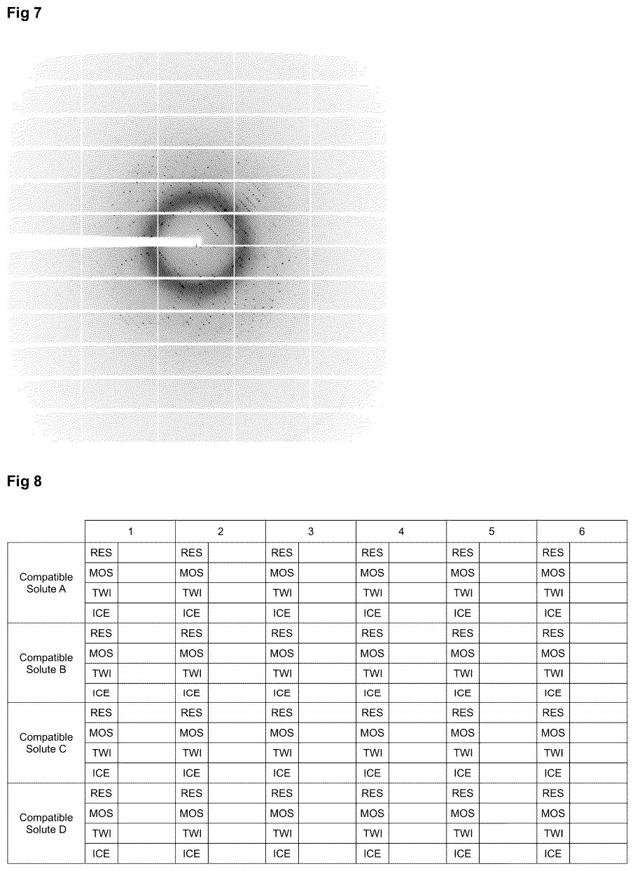

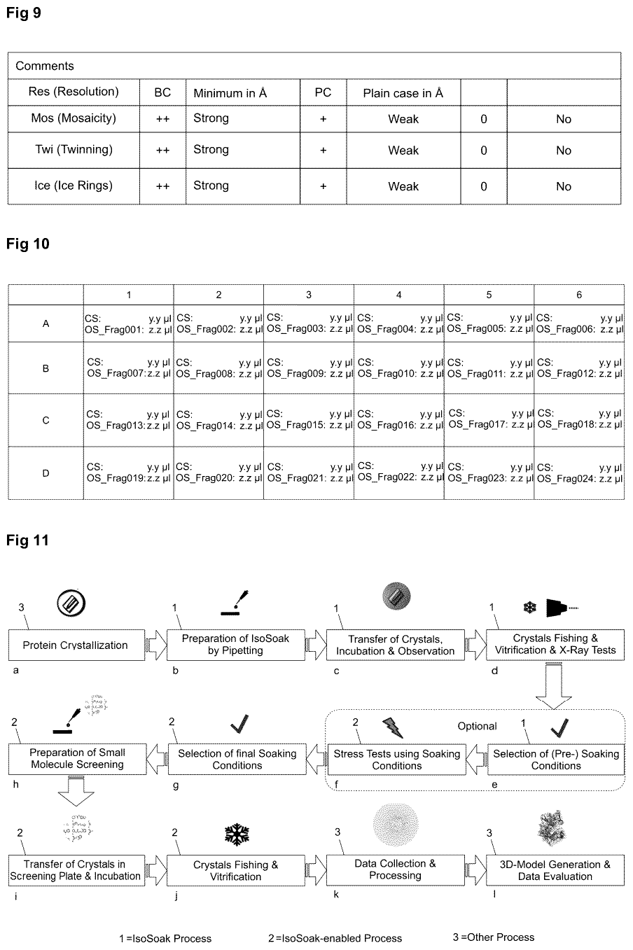

Afterwards, x-ray data is collected. Using an inhouse x-ray source two to several pictures are collected. At a synchrotron source a whole data set is collected, due to its rapid data collection. The raw data (FIG. 7) is evaluated regarding resolution, and mosaicity, the appearance of artifact like data originating from unwanted ice crystals in the protein crystal and sometimes a phenomenon called “twinning”. A template for recording respective results that maps the essential results directly to the experimental layout is exemplified in FIG. 8. In case of data collected at a synchrotron, the appearance of the so called “electron density” is judged by an experienced crystallographer as suitable or not suitable. Based on this data a decision is made on which soaking condition or conditions is/are to be chosen for a Non aqueous soaking solution-based application (step e/g of workflow in FIG. 11). Non aqueous soaking solution can result in a spectrum of soaking conditions. From this spectrum the experimenter can choose alternative water-free solutions according to the goal of the application.

In case of an application for soaking hydrophobic small molecules in a water-free environment at this stage of the workflow optional stress tests can be conducted (step f of workflow in FIG. 11). For this purpose, one or more of the previously obtained water-free environment(s) is/are selected and prepared in a new 24-well plate. Afterwards, crystals are transferred to these solutions. Instead of pure a pure organic solvent, for example DMSO, molecular probes or small molecule fragments for stress tests dissolved in DMSO are used. They exert different kinds of stress such as pH-stress or osmotic stress on the crystals. Based on the subsequently performed data evaluation (same as in step d of workflow in FIG. 11) one or more final soaking solutions can be chosen. The subsequent x-ray analysis can take place using vitrified crystals, crystals in liquid environment under room temperature or dried crystals as described above. Usually, a decision is made for a water-free environment that is subsequently used for the water-free screening of hydrophobic small molecule fragments, for water structure analysis, quality improvement etc. For a Non aqueous soaking solution-based screening of a set of hydrophobic small molecule fragment comprising for example x hydrophobic small molecule fragments 24-well plates are prepared by pipetting the respective water-free soaking solution composition of the chosen soaking solution without DMSO. Instead of pure DMSO, x small molecule species at concentrations of usually 1M dissolved in DMSO are used and added to the prepared wells. This results in final concentrations of the small molecule fragments in each soaking solution of usually 100 mM but may be higher or lower (FIG. 10; step h of workflow in FIG. 11). Following that, usually two newly prepared crystals (sometimes just one or more than two) are transferred to each soaking solution and soaked for usually 24 hours (step i of workflow in FIG. 11). If necessary, the crystals may or may not be conditioned beforehand in respective soaking conditions as described above. Then, the crystals are fished, transferred to liquid nitrogen for vitrification (step j of workflow in FIG. 11), stored in cryo vials, Uni-pucks or some other applicable storage device and sent to a synchrotron facility, where data collection takes place (step k of workflow in FIG. 11). In case of x-ray studies at room temperature no vitrification is required. In case of utilizing dried crystals the drying process as described above is required.

If the purpose of the Non aqueous soaking solution application is quality improvement the small molecule screening may be conducted using soaking conditions identified by a (small or big) IsoSoak-screening (with an original crystallization solution or a suitable salt solution as surrogate) or some other means for identification of soaking conditions. Afterwards the crystals are transferred into an according water-free environment containing or not containing respective small molecule fragments in order to dehydrate the crystals in an ordered process. For structure water analysis small molecule fragments may be used or not.

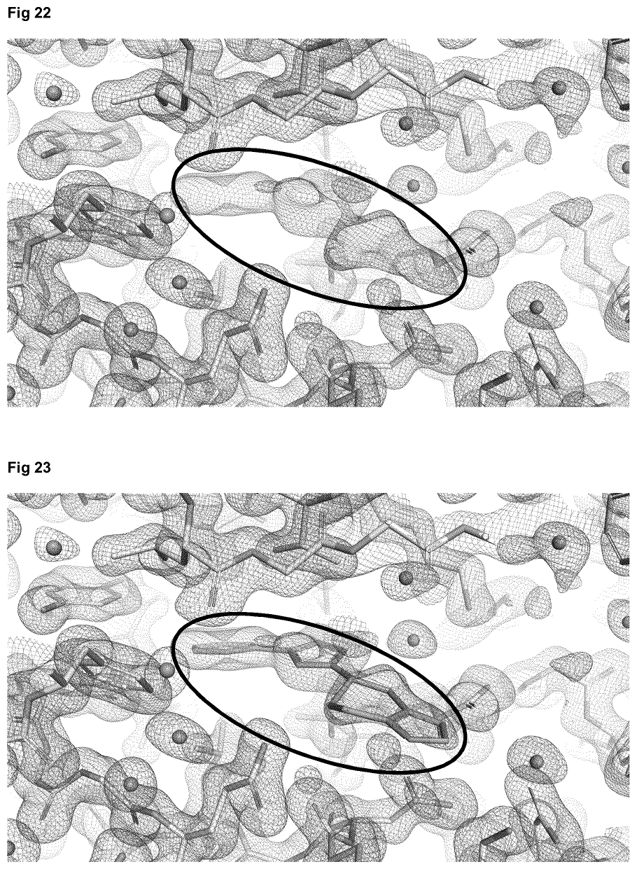

In data collection individual data sets for all crystals are obtained comprising of several hundreds to thousands of pictures showing reflections of the x-ray beams (exemplified in FIG. 7). These reflections stem from interactions between x-rays and electrons and, therefore, can be exploited to reconstruct the so-called electron densities (FIG. 22). Such electron densities define the space in the crystals where electrons are located and are representative for the protein's geometry. Using computational methods important information are extracted from these data sets. To reconstruct the geometry further information is needed, the so-called phases, that are not extractable from this kind of experiment without heavy atom derivatization. This information can be derived in an approach called molecular replacement from previously obtained structural solutions of the same or very similar proteins. The result is a 3-dimensional model of the protein's geometry according to the positions of all the electrons in the protein.

This electron density is merged with the known sequence of amino acids of the respective protein and positions of structurally fixed water molecules are identified (FIG. 23). This solution for the structure is a model that must be optimized in several structure refinement cycles in order to obtain a convincing fit between the measured data and the model. The result is an atomically resolved representation of the geometry of the target (step l of workflow in FIG. 11).

In case of screenings of hydrophobic small molecule fragments each of these structures is examined in order to identify electron density that can be assigned to the special small molecule fragment that was used for soaking of this very crystal. Unassigned electron density appears as is shown in FIG. 24. If this is the case, this small molecule fragment is judged as a so-called “hit”. Examination of all data sets reveals all small molecule fragment-binders. Data sets without additional electron density that is assignable to respective small molecule fragments are discarded. Further refinement of each of these “hit”-data sets reveal the exact geometry of the interaction between the protein and the respective small molecule fragments. Assigning small molecule fragments to unassigned electron densities delivers structures as exemplified in FIG. 25. Therefore, in case of screening of hydrophobic small molecule fragments or other ligands the described process provides validated knowledge about hydrophobic small molecule fragments that bind to the protein target and delivers structurally elucidated binding modes that can be used for medicinal-chemical optimization processes. Instead of small molecule fragments also more drug-size molecules can be used for example to validate follow-up compounds resulting from medicinal-chemical optimization. Furthermore, special molecular probes can be used to map properties of the binding sites or to obtain information about putative pharmacophore patterns etc.

In case of non-aqueous soaking solution-application for quality improvement an according line of action is implemented with the difference of conditioning the crystals from an aqueous soaking-based small molecule fragment screening (using an original crystallization solution or a suitable salt solution as surrogate or any other screening crystal-based approach) in a water-free environment (containing small molecules fragments or not).

In case of structure water analysis small molecule fragments or any other ligand may be used or not. The data analysis after data refinement in this case is focused on differences in observable patterns of water when comparing crystals from aqueous environments and crystals from according water-free environments.

1. A method of selecting the composition of a non-aqueous soaking solution suitable for soaking the crystal form of a biological macromolecule wherein the solution comprises an organic solvent (os) and a compatible solute (cs), said method comprising:
1) preparing the soaking solution by mixing os and cs
2) transferring at least one crystal of the biological macromolecular crystal into the solution prepared in step 1) or transferring each solution prepared in step 1) into a compartment comprising at least one crystal of the biological macromolecular crystal
3) controlling the crystal
4) selecting the soaking solution which is suitable for soaking the crystal; and
wherein the compatible solute is selected polyols, amino acids, methylamines, and mixtures thereof, and
wherein the organic solvent is selected from liquid carbohydrates, protic or aprotic, of low reactivity, that can serve to solve small molecules and, mixtures thereof.

2. The method according to claim 1, wherein said soaking solution comprises an organic solvent (os) and a compatible solute (cs),
wherein said compatible solute (cs) is a polyol or a mixture thereof, and said organic solvent (os) is a liquid carbohydrate protic or aprotic, of low reactivity, or a mixture thereof, that can serve to solve small molecules,
more preferably, said compatible solute (cs) is a poly(oxyethylene) and said organic solvent (os) is selected from a (alkylated) sulfoxide, cyclic non-aromatic ether, straight or branched chain monohydric aliphatic alcohol, alkylated formamide or a mixture thereof,
most preferably, the compatible solute cs is selected from the poly(oxyethylene)s polyethylene glycol 200, polyethylene glycol 400, polyethylene glycol 600, or a mixture thereof and said organic solvent (os) is selected from dimethyl sulfoxide, 1,4-dioxane, dimethylformamide, methanol, ethanol, or a mixture thereof.

3. The method according to claim 1, wherein said soaking solution comprises an organic solvent (os) and a compatible solute (cs),
wherein said compatible solute (cs) is a polyol or a mixture thereof, and said organic solvent is a is a liquid carbohydrate protic or aprotic, of low reactivity, or a mixture thereof, that can serve to solve small molecules,
more preferably, said compatible solute (cs) is a polyhydric alcohol and said organic solvent (os) is selected from a (alkylated) sulfoxide, cyclic non-aromatic ether, straight or branched chain monohydric aliphatic alcohol, alkylated formamide or a mixture thereof,
most preferably, the compatible solute (cs) is selected from the polyhydric alcohols propane-1,2,3-triol (glycerol), ethane-1,2-diol (ethylene glycol), 2-Methylpentane-2,4-diol (MPD), (3R,4S,5S,6R)-2-(2,3-dihydroxypropoxy)-6-(hydroxymethyl)oxane-3,4,5-triol (1-Glucosylglycerol) or a mixtures thereof, and said organic solvent (os) is selected from dimethyl sulfoxide, 1,4-dioxane, dimethylformamide, methanol, ethanol, or a mixture thereof.

4. The method according to claim 1, wherein said soaking solution comprises an organic solvent (os) and a compatible solute (cs),
wherein said compatible solute (cs) is a polyol a or a mixture thereof, and said organic solvent (os) is a liquid carbohydrate protic or aprotic, of low reactivity, or a mixture thereof, that can serve to solve small molecules,
more preferably said compatible solute (cs) is a mono-, di-, tri, oligo- or polysaccharide or a mixture thereof and said organic solvent (os) is selected from a (alkylated) sulfoxide, cyclic non-aromatic ether, straight or branched chain monohydric aliphatic alcohol, alkylated formamide or a mixture thereof,
most preferably, the compatible solute (cs) is selected from the saccharides (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) or β-D-Fructofuranosyl α-D-glucopyranoside (sucrose) or a mixtures thereof, and said organic solvent (os) is selected from dimethyl sulfoxide, 1,4-dioxane, dimethylformamide, methanol, ethanol, or a mixture thereof.

5. The method according to claim 1, wherein said soaking solution comprises an organic solvent (os) and a compatible solute (cs),
wherein said compatible solute (cs) is a methylamine or a mixture thereof and said organic solvent (os) is liquid carbohydrate protic or aprotic, of low reactivity, or a mixture thereof that can serve to solve small molecules,
more preferably said compatible solute (cs) is selected from N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is a (alkylated) sulfoxide, cyclic non-aromatic ether, straight or branched chain monohydric aliphatic alcohol, alkylated formamide, or a mixture thereof,
most preferably said compatible solute (cs) is selected from N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is selected from dimethyl sulfoxide, 1,4-dioxane, dimethylformamide, methanol, ethanol, or a mixture thereof.

6. The method according to claim 1, wherein said soaking solution comprises an organic solvent (os) and a compatible solute (cs), and
wherein said compatible solute (cs) is polyethylene glycol 400 and said organic solvent (os) is dimethyl sulfoxide.

7. The method according to claim 1, wherein said soaking solution comprises an organic solvent (os) and a compatible solute (cs),
wherein said compatible solute (cs) is ethane-1,2-diol (ethylene glycol) and said organic solvent (os) is dimethyl sulfoxide.

8. The method according to claim 1, wherein said soaking solution comprises an organic solvent (os) and a compatible solute (cs),
wherein said compatible solute (cs) is propane-1,2,3-triol (glycerol) and said organic solvent (os) is dimethyl sulfoxide.

9. The method according to claim 1, wherein said soaking solution comprises an organic solvent (os) and a compatible solute (cs),
wherein said compatible solute (cs) is 2-methylpentane-2,4-diol (MPD) and said organic solvent (os) is dimethyl sulfoxide.

10. A method of selecting the composition of a non-aqueous soaking solution for soaking the crystal form of a biological macromolecule wherein the soaking solution comprises an organic solvent (os) and a compatible solute (cs); said method comprising:
1) preparing a first series of In to xn individual soaking solutions by mixing os and cs, wherein:
a) x is at least 2
b) each soaking solutions of the first series comprises the same organic solvent (os) and the same compatible solute (cs)
c) Vos and Vcs are changed in each solution of the series, and
in the solution 1n, Vₒₛ is the minimum Vₒₛ (Vₒₛmin) and the V_{cs} is the maximum Vcs (V_{cs}max), and
in the solution xn, V_{cs} is the minimum V_{cs} (V_{cs}min) and the Vₒₛ is the maximum Vₒₛ (Vₒₛmax), and
when x>2, in any additional solutions between 1n and xn, the Vₒₛ is varied between Vₒₛmin and Vₒₛmax and, inversely, Vcs is varied between V_{cs}min and V_{cs}max;
or, alternatively, Vₒₛ and N_{cs} are changed in each solution of the series, and
in the solution 1n, Vₒₛ is the minimum Vₒₛ (Vₒₛmin) and the N_{cs} is the maximum N_{cs} (N_{cs}max), and
in the solution xn, N_{cs} is the minimum N_{cs} (N_{cs}min) and the Vₒₛ is the maximum Vₒₛ (Vₒₛmax), and
when x>2, in any additional solutions between 1n and xn, the Vₒₛ is varied between Vₒₛmin and Vₒₛmax and, inversely, Ncs is varied between N_{cs}min and N_{cs}max,
2) transferring at least one crystal of the biological macromolecular crystal into y compartments, each compartment comprising one of the solutions prepared in step 1 or transferring each solution prepared in step 1 into y compartments, each compartment comprising at least one crystal of the biological macromolecular crystal from its crystallization solution,
3) controlling the crystal in each compartment, and
4) selecting the soaking solution(s) which is/are suitable for soaking the crystal.

11. of the method according to claim 10, further comprising:
1) preparing m series of In to yn individual soaking solutions by mixing os and cs wherein m is at least 1 and wherein in each of the m series
a) y is at least 2
b) os is the same as in the first series, and
c) the cs is changed and is different from the cs of the first series, and
wherein in each individual solutions of each of the m series
d) Vₒₛ and Vcs are changed in each solution, and
in the solution 1n, Vₒₛ is the minimum Vₒₛ (Vₒₛmin) and the V_{cs} is the maximum V_{cs} (V_{cs}max), and
in the solution yn, V_{cs} is the minimum V_{cs} (V_{cs}min) and the Vₒₛ is the maximum Vₒₛ (Vₒₛmax), and
when y>2, in any additional solutions between 1n and yn, the Vₒₛ is varied between Vₒₛmin and Vₒₛmax and, inversely, V_{cs} is varied between V_{cs} min and V_{cs} max,
or, alternatively, Vos and Ncs are changed in each solution, and
in the solution 1n, Vₒₛ is the minimum Vₒₛ (Vosmin) and the N_{cs} is the maximum N_{cs} (N_{cs}max), and
in the solution yn, N_{cs} is the minimum N_{cs} (N_{cs}min) and the Vₒₛ is the maximum Vₒₛ (Vₒₛmax), and
when y>2, in any additional solutions between 1n and yn, the Vₒₛ is varied between Vₒₛmin and Vₒₛmax and, inversely, N_{cs} is varied between N_{cs}min and V_{cs} max, and
2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.

12. of the method according to claim 10, further comprising:
1) preparing m series of In to yn individual soaking solutions by mixing os and cs wherein m is at least 1 and wherein in each of the m series
a) y is at least 2
b) cs is the same as in the first series, and
c) the os is changed and is different from the os of the first series, and
wherein in each individual solutions of each of the m series
d) Vₒₛ and Vcs are different in each solution, and
in the solution 1n, Vₒₛ is the minimum Vₒₛ (Vₒₛmin) and the V_{cs} is the maximum V_{cs} (V_{cs}max), and
in the solution yn, V_{cs} is the minimum V_{cs} (V_{cs}min) and the Vₒₛ is the maximum Vₒₛ (Vₒₛmax), and
when y>2, in any additional solutions between 1n and yn, the Vₒₛ varied between Vₒₛmin and Vₒₛmax and, inversely, V_{cs} is varied between V_{cs} min and V_{cs} max,
or, alternatively, Vos and Ncs are different in each solution, and
in the solution 1n, Vₒₛ is the minimum Vₒₛ (Vₒₛmin) and the N_{cs} is the maximum N_{cs} (N_{cs}max), and
in the solution yn, N_{cs} is the minimum N_{cs} (N_{cs}min) and the Vₒₛ is the maximum Vₒₛ (Vₒₛmax), and
when y>2, in any additional solutions between 1n and yn, the Vₒₛ varied between Vₒₛmin and Vₒₛmax and, inversely, N_{cs} is varied between N_{cs}min and N_{cs}max, and
2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.

13. The method according to claim 1, wherein the os contains small molecules including molecule fragments and molecular probes to be analyzed, which diffuse into the biological macromolecular crystal.

14. A method of small molecule screening for a biological macromolecular crystal, comprising performing the screening in a soaking solution selected according to the method of claim 13.

15. A soaking solution obtainable by the method of selecting the composition of a non-aqueous soaking solution according to claim 1.

16. for a method of small molecule screening for a biological macromolecular crystal, comprising performing the screening in a soaking solution according to claim 15.

17. An array arranged to perform the method according to claim 1, wherein said array comprises a first dimension of at least two individual non-aqueous soaking solutions (1n to xn) and a second dimension of at least two individual non-aqueous soaking solutions (1m to ym) wherein each of said soaking solutions is located in a separated compartment of said array, and wherein each of said soaking solution comprises an organic solvent (os) and a compatible solute (cs).

18. A method of obtaining a soaking solution comprising using an array according to claim 17.
